SEQUENCE LISTING (14 sequences)

SEQ ID NO: 1 - DNA, 23 bp, Artificial
  Luciferase siRNA
cauucuauccucuagaggaugtt

SEQ ID NO: 2 - DNA, 21 bp, artificial
  Chk1 siRNA
gaagcagucgcagugaagatt

SEQ ID NO: 3 - RNA, 21 nt, artificial
  Pol alpha siRNA
gcaguaacaucgauuguaauu

SEQ ID NO: 4 - RNA, 21 nt, artificial
  Pol epsilon siRNA
agagaaggcuggcggauuauu

SEQ ID NO: 5 - RNA, 21 nt, artificial
  Pol delta siRNA
ccgacgugaucaccgguuauu

SEQ ID NO: 6 - RNA, 21 nt, artificial
  ATR siRNA
ggucagcugucuacuguuauu

SEQ ID NO: 7 - RNA, 21 nt, artificial
  ATM siRNA
aggaggagcuugggccuuuuu

SEQ ID NO: 8 - protein, 27 aa, Homo sapiens
CNRERLLNKMCGTLPYVAPELLKRREF

SEQ ID NO: 9 - protein, 18 aa, Homo sapiens
  MISC_FEATURE (18)..(18): C-terminal Biotin
RSGLYRSPSMPENLNRPR

SEQ ID NO: 10 - protein, 8 aa, artificial
  Hemagglutinin Epitope Tag
YDVPDYAS

SEQ ID NO: 11 - DNA, 5448 bp, Homo sapiens
  CDS (24)..(4412)
gcggaatcgggagattcgggaccatggcacctgtgcacggcgacgactctctgtcagattcagggagttttgtatcttctcgagcccggcgagaaaaaaaatcaaagaaggggcgccaagaagccctagaaagactgaaaaaggctaaagctggtgagaagtataaatatgaagtcgaggacttcacaggtgtttatgaagaagttgatgaagaacagtattcgaagctggttcaggcacgccaggatgatgactggattgtggatgatgatggtattggctatgtggaagatggccgagagatttttgatgatgaccttgaagatgatgcccttgatgctgatgagaaaggaaaagatggtaaagcacgcaataaagacaagaggaatgtaaagaagctcgcagtgacaaaaccgaacaacattaagtcaatgttcattgcttgtgctggaaagaaaactgcagataaagctgtagacttgtccaaggatggtctgctaggtgacattctacaggatcttaacactgagacacctcaaataactccaccacctgtaatgatactgaagaagaaaagatccattggagcttcaccgaatcctttctctgtgcacaccgccacggcagttccttcaggaaaaattgcttcccctgtctccagaaaggagcctccattaactcctgttcctcttaaacgtgctgaatttgctggcgatgatgtacaggtcgagagtacagaagaagagcaggagtcaggggcaatggagtttgaagatggtgactttgatgagcccatggaagttgaagaggtggacctggagcctatggctgccaaggcttgggacaaagagagtgagccagcagaggaagtgaaacaagaggcggattctgggaaagggaccgtgtcctacttaggaagttttctcccggatgtctcttgttgggacattgatcaagaaggtgatagcagtttctcagtgcaagaagttcaagtggattccagtcacctcccattggtaaaaggggcagatgaggaacaagtattccacttttattggttggatgcttatgaggatcagtacaaccaaccaggtgtggtatttctgtttgggaaagtttggattgaatcagccgagacccatgtgagctgttgtgtcatggtgaaaaatatcgagcgaacgctttacttccttccccgtgaaatgaaaattgatctaaatacggggaaagaaacaggaactccaatttcaatgaaggatgtttatgaggaatttgatgagaaaatagcaacaaaatataaaattatgaagttcaagtctaagccagtggaaaagaactatgcttttgagatacctgatgttccagaaaaatctgagtacttggaagttaaatactcggctgaaatgccacagcttcctcaagatttgaaaggagaaactttttctcatgtatttgggaccaacacatctagcctggaactgttcttgatgaacagaaagatcaaaggaccttgttggcttgaagtaaaaagtccacagctcttgaatcagccagtcagttggtgtaaagttgaggcaatggctttgaaaccagacctggtgaatgtaattaaggatgtcagtccaccaccgcttgtcgtgatggctttcagcatgaagacaatgcagaatgcaaagaaccatcaaaatgagattattgctatggcagctttggtccatcacagttttgcattggataaagcagccccaaagcctccctttcagtcacacttctgtgttgtgtctaaaccaaaggactgtatttttccatatgctttcaaagaagtcattgagaaaaagaatgtgaaggttgaggttgctgcaacagaaagaacactgctaggttttttccttgcaaaagttcacaaaattgatcctgatatcattgtgggtcataatatttatgggtttgaactggaagtactactgcagagaattaatgtgtgcaaagctcctcactggtccaagataggtcgactgaagcgatccaacatgccaaagcttgggggccggagtggatttggtgaaagaaatgctacctgtggtcgaatgatctgtgatgtggaaatttcagcaaaggaattgattcgttgtaaaagctaccatctgtctgaacttgttcagcagattctaaaaactgaaagggttgtaatcccaatggaaaatatacaaaatatgtacagtgaatcttctcaactgttatacctgttggaacacacctggaaagatgccaagttcattttgcagatcatgtgtgagctaaatgttcttccattagcattgcagatcactaacatcgctgggaacattatgtccaggacgctgatgggtggacgatccgagcgtaacgagttcttgttgcttcatgcattttacgaaaacaactatattgtgcctgacaagcagattttcagaaagcctcagcaaaaactgggagatgaagatgaagaaattgatggagataccaataaatacaagaaaggacgtaagaaagcagcttatgctggaggcttggttttggaccccaaagttggtttttatgataagttcattttgcttctggacttcaacagtctatatccttccatcattcaggaatttaacatttgttttacaacagtacaaagagttgcttcagaggcacagaaagttacagaggatggagaacaagaacagatccctgagttgccagatccaagcttagaaatgggcattttgcccagagagatccggaaactggtagaacggagaaaacaagtcaaacagctaatgaaacagcaagacttaaatccagaccttattcttcagtatgacattcgacagaaggctttgaagctcacagcgaacagtatgtatggttgcctgggattttcctatagcagattttacgccaaaccactggctgccttggtgacatacaaaggaagggagattttgatgcatacgaaagagatggtacaaaagatgaatcttgaagttatttatggagatacagattcaattatgataaacaccaatagcaccaatctggaagaagtatttaagttgggaaacaaggtaaaaagtgaagtgaataagttgtacaaactgcttgaaatagacattgatggggttttcaagtctctgctactgctgaaaaaaaagaagtacgctgctctggttgttgagccaacgtcggatgggaattatgtcaccaaacaggagctcaaaggattagatatagttagaagagattggtgtgatcttgctaaagacactggaaactttgtgattggccagattctttctgatcaaagccgggacactatagtggaaaacattcagaagaggctgatagaaattggagaaaatgtgctaaatggcagtgtcccagtgagccagtttgaaattaacaaggcattgacaaaggatccccaggattaccctgataaaaaaagcctacctcatgtacatgttgccctctggataaattctcaaggaggcagaaaggtgaaagctggagatactgtgtcatatgtcatctgtcaggatggatcaaacctcactgcaagtcagagggcctatgcgcctgagcagctgcagaaacaggataatctaaccattgacacccagtactacctggcccagcagatccacccagtcgtggctcggatctgtgaaccaatagacggaattgatgctgtcctcattgcaacgtggttgggacttgaccccacccaatttagagttcatcattatcataaagatgaagagaatgatgctctacttggtggcccagcacagctcactgatgaagagaaatacagggactgtgaaagattcaaatgtccatgccctacatgtggaactgagaatatttatgataatgtctttgatggttcgggaacagatatggagcccagcttgtatcgttgcagtaacatcgattgtaaggcttcacctctgacctttacagtacaactgagcaacaaattgatcatggacattagacgtttcattaaaaagtactatgatggctggttgatatgtgaagagccaacctgtcgcaatcgaactcgtcaccttccccttcaattctcccgaactgggcctctttgcccagcctgcatgaaagctacacttcaaccagagtattctgacaagtccctgtacacccagctgtgcttttaccggtacatttttgatgcggagtgtgcactggagaaacttactaccgatcatgagaaagataaattgaagaagcaattttttacccccaaagttctgcaggactacagaaaactcaagaacacagcagagcaattcttgtcccgaagtggctactccgaagtgaatctgagcaaactcttcgctggttgtgccgtgaaatcctaagggaatcccaggagtaaccaaggagggggtagttgaaaaatcccagcttcctctgtgcctccactctggccctaaatgctcctccagcatctgtttctcccttgggactgtgtctcatgtttgtgtgaatgtagaccaggaaagggggctgcaaaaatgttgagtctaatgttcgtaagcatcatagaaattcctgtcttcatattaagatgtactgctttaaaacacaactccagagcccctccccaagctcccctccccaagctcctgaagacccggtttctgagggagggaaattgctacttggattgagagtagctggaatgtaagtgaccccaggctttgcctcagggcctttagcctatgtcccccccacataaagagagcttctcagagcctgactgaagagctgacgttttgctttttcatatgccaattaaacccggtctaaatccaaatgcttctccagccatccaggagtggctgtccttttcagtcttgtcttttatataggtagctgagggggaagatttagaagccttgcactcactaaatagattaaacagagcaggcttgtttgttgaattgctccaaagtccaacagacacacactgagcaggtgttttacactcacattccctttttgccccttaaatagaaagtgcaggtaaaggtttatacaacaagaaagcacattgaaaataatttgatactctaacaatccattaacatgtgtaggggttacggtgaggatcactgtgttgtattcagaaaaacggggagagggatgcttaattggccctggcgcttgctatttttttctcatttcttcacaataggaccgtctttggcagcagcaaaatgtatttcagtatggcagtctttcctctcttacattattggtaagattatactaacaaaatgtttccccttgtacaattatgctgtgtttttaaaaaacattgacctgtgtgtttttataaaagaaaaagtatgttgtgccttcttcttaagaataaagttttctaaagggaaa

SEQ ID NO: 12 - protein, 1462 aa, Homo sapiens
MAPVHGDDSLSDSGSFVSSRARREKKSKKGRQEALERLKKAKAGEKYKYEVEDFTGVYEEVDEEQYSKLVQARQDDDWIVDDDGIGYVEDGREIFDDDLEDDALDADEKGKDGKARNKDKRNVKKLAVTKPNNIKSMFIACAGKKTADKAVDLSKDGLLGDILQDLNTETPQITPPPVMILKKKRSIGASPNPFSVHTATAVPSGKIASPVSRKEPPLTPVPLKRAEFAGDDVQVESTEEEQESGAMEFEDGDFDEPMEVEEVDLEPMAAKAWDKESEPAEEVKQEADSGKGTVSYLGSFLPDVSCWDIDQEGDSSFSVQEVQVDSSHLPLVKGADEEQVFHFYWLDAYEDQYNQPGVVFLFGKVWIESAETHVSCCVMVKNIERTLYFLPREMKIDLNTGKETGTPISMKDVYEEFDEKIATKYKIMKFKSKPVEKNYAFEIPDVPEKSEYLEVKYSAEMPQLPQDLKGETFSHVFGTNTSSLELFLMNRKIKGPCWLEVKSPQLLNQPVSWCKVEAMALKPDLVNVIKDVSPPPLVVMAFSMKTMQNAKNHQNEIIAMAALVHHSFALDKAAPKPPFQSHFCVVSKPKDCIFPYAFKEVIEKKNVKVEVAATERTLLGFFLAKVHKIDPDIIVGHNIYGFELEVLLQRINVCKAPHWSKIGRLKRSNMPKLGGRSGFGERNATCGRMICDVEISAKELIRCKSYHLSELVQQILKTERVVIPMENIQNMYSESSQLLYLLEHTWKDAKFILQIMCELNVLPLALQITNIAGNIMSRTLMGGRSERNEFLLLHAFYENNYIVPDKQIFRKPQQKLGDEDEEIDGDTNKYKKGRKKAAYAGGLVLDPKVGFYDKFILLLDFNSLYPSIIQEFNICFTTVQRVASEAQKVTEDGEQEQIPELPDPSLEMGILPREIRKLVERRKQVKQLMKQQDLNPDLILQYDIRQKALKLTANSMYGCLGFSYSRFYAKPLAALVTYKGREILMHTKEMVQKMNLEVIYGDTDSIMINTNSTNLEEVFKLGNKVKSEVNKLYKLLEIDIDGVFKSLLLLKKKKYAALVVEPTSDGNYVTKQELKGLDIVRRDWCDLAKDTGNFVIGQILSDQSRDTIVENIQKRLIEIGENVLNGSVPVSQFEINKALTKDPQDYPDKKSLPHVHVALWINSQGGRKVKAGDTVSYVICQDGSNLTASQRAYAPEQLQKQDNLTIDTQYYLAQQIHPVVARICEPIDGIDAVLIATWLGLDPTQFRVHHYHKDEENDALLGGPAQLTDEEKYRDCERFKCPCPTCGTENIYDNVFDGSGTDMEPSLYRCSNIDCKASPLTFTVQLSNKLIMDIRRFIKKYYDGWLICEEPTCRNRTRHLPLQFSRTGPLCPACMKATLQPEYSDKSLYTQLCFYRYIFDAECALEKLTTDHEKDKLKKQFFTPKVLQDYRKLKNTAEQFLSRSGYSEVNLSKLFAGCAVKS

SEQ ID NO: 13 - DNA, 2042 bp, Homo sapiens
  CDS (159)..(1589)
ggggccagtcgttcgccggaaagcatttgtctcccacctcatcataacaacaattaatttcctctggggcctgaggagggcagaatttcaaccttcggtgtgcttgggagtggcgattgtgatttacacgacaaaatgccgaggtgctcggtggagtcatggcagtgccctttgtggaagactgggacttggtgcaaaccctgggagaaggtgcctatggagaagttcaacttgctgtgaatagagtaactgaagaagcagtcgcagtgaagattgtagatatgaagcgtgccgtagactgtccagaaaatattaagaaagagatctgtatcaataaaatgctaaatcatgaaaatgtagtaaaattctatggtcacaggagagaaggcaatatccaatatttatttctggagtactgtagtggaggagagctttttgacagaatagagccagacataggcatgcctgaaccagatgctcagagattcttccatcaactcatggcaggggtggtttatctgcatggtattggaataactcacagggatattaaaccagaaaatcttctgttggatgaaagggataacctcaaaatctcagactttggcttggcaacagtatttcggtataataatcgtgagcgtttgttgaacaagatgtgtggtactttaccatatgttgctccagaacttctgaagagaagagaatttcatgcagaaccagttgatgtttggtcctgtggaatagtacttactgcaatgctcgctggagaattgccatgggaccaacccagtgacagctgtcaggagtattctgactggaaagaaaaaaaaacatacctcaacccttggaaaaaaatcgattctgctcctctagctctgctgcataaaatcttagttgagaatccatcagcaagaattaccattccagacatcaaaaaagatagatggtacaacaaacccctcaagaaaggggcaaaaaggccccgagtcacttcaggtggtgtgtcagagtctcccagtggattttctaagcacattcaatccaatttggacttctctccagtaaacagtgcttctagtgaagaaaatgtgaagtactccagttctcagccagaaccccgcacaggtctttccttatgggataccagcccctcatacattgataaattggtacaagggatcagcttttcccagcccacatgtcctgatcatatgcttttgaatagtcagttacttggcaccccaggatcctcacagaacccctggcagcggttggtcaaaagaatgacacgattctttaccaaattggatgcagacaaatcttatcaatgcctgaaagagacttgtgagaagttgggctatcaatggaagaaaagttgtatgaatcaggttactatatcaacaactgataggagaaacaataaactcattttcaaagtgaatttgttagaaatggatgataaaatattggttgacttccggctttctaagggtgatggattggagttcaagagacacttcctgaagattaaagggaagctgattgatattgtgagcagccagaaggtttggcttcctgccacatgatcggaccatcggctctggggaatcctggtgaatatagtgctgctatgttgacattattcttcctagagaagattatcctgtcctgcaaactgcaaatagtagttcctgaagtgttcacttccctgtttatccaaacatcttccaatttattttgtttgttcggcatacaaataatacctatatcttaattgtaagcaaaactttggggaaaggatgaatagaattcatttgattatttcttcatgtgtgtttagtatctgaatttgaaactcatctggtggaaaccaagtttcaggggacatgagttttccagcttttatacacacgtatctcatttttatcaaaacattttgtttaattcaaaaagtacatatttcttccatgttgatttaattctaagatgaaccaataaagacataattcttgcaaaaaaaaaaaaaaaaaaaaaaaaaa

SEQ ID NO: 14 - protein, 476 aa, Homo sapiens
MAVPFVEDWDLVQTLGEGAYGEVQLAVNRVTEEAVAVKIVDMKRAVDCPENIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASSEENVKYSSSQPEPRTGLSLWDTSPSYIDKLVQGISFSQPTCPDHMLLNSQLLGTPGSSQNPWQRLVKRMTRFFTKLDADKSYQCLKETCEKLGYQWKKSCMNQVTISTTDRRNNKLIFKVNLLEMDDKILVDFRLSKGDGLEFKRHFLKIKGKLIDIVSSQKVWLPAT

(19) United States
(12) Patent Application Publication
(10) Pub. No.: US 2010/0143332 A1
(43) Pub. Date: Jun. 10, 2010

(54) COMBINATION THERAPY FOR PROLIFERATIVE DISORDERS

(75) Inventors: David A. Parry, Mountain View, CA (US); Lorena Taricani, Campbell, CA (US)

Correspondence Address: MERCK;C/O SCHERING-PLOUGH BIOPHARMA, LEGAL DEPARTMENT, 901 CALIFORNIA AVENUE, PALO ALTO, CA, 94304, US

(73) Assignee: Schering Corporation

(21) Appl. No.: 12/514,758

(22) PCT Filed: Nov. 15, 2007
(86) PCT No.: PCT/US07/24064

§ 371 Date: Jan. 14, 2010

Related U.S. Application Data
(60) Provisional application No. 60/859,760, filed on Nov. 17, 2006.

(51) Int. Cl. A61K 39/395; A61K 31/7088

(52) U.S. Cl. 424/130.1; 514/44 A

(57) ABSTRACT

The present invention provides compositions and methods of treating proliferative disorders using combination therapy with a first agent that specifically inhibits DNA polymerase alpha and a second agent that inhibits protein kinases, such as Chk1.

### FIELD OF THE INVENTION

The present invention relates to methods and compositions for treatment of proliferative disorders, such as cancer. Specifically, the invention relates to combination therapy with a first agent that interferes with DNA replication and a second agent that interferes with a replication checkpoint.

### BACKGROUND OF THE INVENTION

Complex networks of surveillance mechanisms, referred to as “checkpoints”, maintain genomic integrity in the face of various genomic insults (Hartwell & Weinert (1989) Science 246:629; Weinert (1997) Science 277:1450; Kastan & Bartek (2004) Nature 432:316). Checkpoint kinases (e.g. Chk1, Chk2 etc.) prevent cell cycle progression at inappropriate times, such as in response to DNA damage, and maintain the metabolic balance of cells while the cell is arrested, and in some instances can induce apoptosis (programmed cell death) when the requirements of the checkpoint have not been met. Checkpoint control can occur in the G1 phase, prior to DNA synthesis (the “G1/S checkpoint”), in S-phase (the “intra-checkpoint”) and in G2, prior to entry into mitosis (the “G2/M checkpoint”). This action enables DNA repair processes to complete their tasks before replication of the genome and subsequent separation of this genetic material into new daughter cells takes place. Inactivation of CHK1 has been shown to abrogate the G2 arrest that would normally be induced by DNA damage (endogenous DNA damage or damage caused by anticancer agents), resulting in inappropriate mitotic entry and preferential killing of the resulting checkpoint defective cells. See, e.g., Peng et al. (1997) Science, 277:1501; Sanchez et al. (1997) Science 277:1497; Nurse (1997) Cell 91:865; Weinert (1997) Science 277:450; Walworth et al. (1993) Nature 363:368; and Al-Khodairy et al. (1994) Molec. Biol. Cell 5:147. These effects are thought to be mediated by the role of Chk1 in the regulation of the activity of Cdc25C, which in turn regulates the activity of the Cdc-2/cyclinB complex that regulates mitotic entry.

Chk1, a serine/threonine checkpoint kinase, contributes to both intra-S and G2/M checkpoint responses (Liu et al. (2000) Genes Dev. 14:1448; Sorensen et al. (2003) Cancer Cell 3:247; Cho et al. (2005) Cell Cycle 4:131; Zachos et al. (2005) Mol. Cell. Biol. 25:563; Petermann et al. (2006) Mol. Cell. Biol. 26:3319). Following replication stress and engagement of the intra-S checkpoint, Chk1 is activated by ATM (ataxia telangiectasia, mutated) and ATR (ATM and Rad3-related) protein kinases. It has been shown that exposure to DNA antimetabolite drugs activates the intra-S checkpoint (see, e.g., Cho et al. (2005) Cell Cycle 4:131) but the mechanism by which Chk1 contributes to this response remains unclear.

Chk1 inhibitors have been proposed as potentially useful adjuncts to cancer therapy using chemotherapeutic agents. See, e.g., Tao & Lin (2006) Anti-Cancer Agents in Med. Chem. 6:377. Inhibition of the activity of Chk1 is predicted to lead to failure of checkpoint regulation in cancer cells harboring chemotherapy-induced DNA damage. Checkpoint failure leads to progression of cells into mitosis despite DNA damage, leading to mitotic crisis and ultimately apoptosis. Non-cancerous cells are predicted to be less sensitive to the loss of Chk1-mediated checkpoint function since they are generally less rapidly dividing, and they might also have functional G1 checkpoint (lacking in most tumor cells) to prevent progression through the cell cycle into mitosis. This differential effect of Chk1 inhibitors on cancerous versus normal cells is predicted to enhance the effectiveness of chemotherapy and provide greater tumor killing for a given level of undesirable side effects.

Checkpoint inhibitors, such as caffeine, UCN-01, Gö6979, ICP-1, SB218078, PD166285 and isogranulatimide have been combined with DNA-damaging agents or radiation, as reviewed in Prudhomme (2004) Curr. Med. Chem.—Anti-Cancer Agents 4:435. Inhibition of Chk1 has been combined with nucleoside analogs (Sampath et al. (2003) Oncogene 22:9063) and other chemotherapeutic agents and antimetabolites, such as etoposide, doxorubicin, cisplatin, chlorambucil, 5-fluorouracil, methotrexate, hydroxyurea, 2-chloroadenosine, fludarabine, azacytidine, gemcitibine (U.S. Pat. Nos. 7,067,506; U.S. Pat. App. Publication Nos. 2003/0069284; and WO 2005/027907), cytosine arabinoside (ara-C) and thymidine (Cho et al. (2005) Cell Cycle 4:131), aphidicolin (Zachos et al. (2003) EMBO J.: 22:713), and 7-hydroxystaurosporine (UCN-01) (Feijoo et al. (2001) J. Cell Biol. 154:913; Miao et al. (2003) J. Biol. Chem. 278:4295).

The need exists for improved methods to preferentially sensitize tumor tissues, as opposed to normal tissues, to the toxic effects of chemotherapeutic agents and DNA antimetabolites, in order to facilitate the treatment or prevention of disease states associated with abnormal cell proliferation. Preferably, such methods and compositions should induce mitotic crisis or apoptosis in tumor tissues. Preferably such methods and compositions would also be highly selective for tumor tissue, thus minimizing undesirable side-effects. Preferably, such methods and compositions would be narrowly targeted to inhibit only the molecules (e.g. a specific DNA polymerase) absolutely necessary to achieve the therapeutic benefit, while being less disruptive to other molecules, thus minimizing undesirable side-effects. Preferably, such methods and compositions involve compounds that are not incorporated into DNA, providing for prolonged arrest of DNA synthesis, enhanced activation of the DNA checkpoint, and increased effectiveness of checkpoint inhibitors as therapeutic agents.

### SUMMARY OF THE INVENTION

In its many embodiments, the present invention provides methods of treatment of proliferative disorders involving inhibiting the activity of DNA polymerase alpha and inhibiting the activity of at least one checkpoint kinase, e.g. Chk1. In another aspect, the present invention provides methods of treatment of proliferative disorders in a subject, e.g. a subject in need thereof, by administering to the subject a first agent that is an inhibitor of DNA polymerase alpha and a second agent that is an inhibitor of at least one checkpoint kinase, e.g. Chk1 or Chk2. In yet another aspect, the invention relates to a composition that is administered to a subject in need thereof, comprising an inhibitor of DNA polymerase alpha and an inhibitor of a checkpoint kinase, e.g. Chk1 or Chk2.

In some embodiments of all aspects of the invention the checkpoint kinase is Chk1.

In various embodiments, the first agent is administered prior to, concurrently with, or subsequent to the second agent. In other embodiments, treatment with said first and/or second agents is repeated more than once, in any sequence. In a preferred embodiment, said first agent is administered at a first time, and said second agent is administered at a later time, at which later time administration of said first compound may be continued or discontinued.

In some embodiments, the inhibition of DNA polymerase alpha is at least 1.5-, 2-, 3-, 4-, 5-, 6-, 7-, 8-, 9-, 10-, 12-, 15-, 20-, 25-, 30-, 40-, 50-, 60-, 70-, 80-, 90-, 100-, 150-, 200-, 300-, 400-, 500-, 700-, 1000-fold or more greater than the inhibition of another DNA polymerase, e.g. DNA polymerase epsilon.

Exemplary first agents include, but are not limited to, 4-hydroxy-17-methylincisterol, the glycolipid galactosyldiacylglycerol (GDG), the paclitaxel derivative cephalomannine, dehydroaltenusin, sulfolipid compounds (e.g. sulfoquinovosyldiacylglycerol), acyclic phosphonmethoxyalkyl nucleotide analogs, resveratrol (3,4,5-trihydroxystilbene), the triterpene dicarboxylic acid mispyric acid, 6-(p-n-butylanilino)uracil and N2-(p-butylphenyl)guanine.

In one embodiment, the first agent is selected from the group consisting of 4-hydroxy-17-methylincisterol, galactosyldiacylglycerol, cephalomannine, dehydroaltenusin, 6-(p-n-butylanilino)uracil and N2-(p-butylphenyl)guanine. In another embodiment, the first agent is cephalomannine. In yet another embodiment the first agent is dehydroaltenusin.

Exemplary second agents include, but are not limited to, pyrazolopyrimidines, imidazopyrazines, UCN-01, indolcarbazole compounds, Gö6976, SB-218078, staurosporine, ICP-1, CEP-3891, isogranulatimide, debromohymenialdisine (DBH), pyridopyrimidine derivatives, PD0166285, scytonemin, diaryl ureas, benzimidazole quinolones, CHR 124, CHR 600, tricyclic diazopinoindolones, PF-00394691, furanopyrimidines, pyrrolopyrimidines, indolinones, substituted pyrazines, compound XL844, pyrimidinylindazolyamines, aminopyrazoles, 2-ureidothiophenes, pyrimidines, pyrrolopyrimidines, 3-ureidothiophenes, indenopyazoles, triazlones, dibenzodiazepinones, macrocyclic ureas, pyrazoloquinoloines, and the peptidomimetic CBP501. In one embodiment the second agent is selected from the group consisting of a pyrazolopyrimidine or an imidazopyrazine. In another embodiment, the pyrazolopyrimidine is a pyrazolo[1,5-a]pyrimidine. In another embodiment, the imidazopyrazine is an imidazo[1,2-a]pyrazine.

In some embodiments one or more additional agents is included in combination with said first and second agents, such as one or more anti-cancer agent selected from the group consisting of a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, Iressa®, Tarceva®, antibodies to EGFR, Gleevec®, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin (Eloxatin®), leucovirin, pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17a-ethinylestradiol, diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, Avastin®, Herceptin® (trastuzumab), Bexxar®, Velcade®, Zevalin®, Trisenox®, Xeloda®, vinorelbine, porfimer, Erbitux®, liposomal, thiotepa, altretamine, melphalan, lerozole, fulvestrant, exemestane, fulvestrant, ifosfomide, C225, Campath®, clofarabine, cladribine, aphidicolon, Rituxan® (rituximab), sunitinib, dasatinib, tezacitabine, 5 ml 1, fludarabine, pentostatin, Triapine®, didox, trimidox, amidox, 3-AP, and MDL-101,731.

In some embodiments, the proliferative disorder is cancer, autoimmune disease, viral disease, fungal disease, neurological/neurodegenerative disorder, arthritis, inflammation, anti-proliferative disease, neuronal disease, alopecia, cardiovascular disease or sepsis.

In one embodiment the proliferative disorder is cancer. In some embodiments the cancer is selected from the group consisting of cancer of the bladder, breast, colon, kidney, liver, lung, small cell lung cancer, non-small cell lung cancer, head and neck, esophagus, gall bladder, ovary, pancreas, stomach, cervix, thyroid, prostate, and skin, squamous cell carcinoma; leukemia, acute lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, Burkett's lymphoma; acute and chronic myelogenous leukemia, myelodysplastic syndrome, promyelocytic leukemia; fibrosarcoma, rhabdomyosarcoma; astrocytoma, neuroblastoma, glioma and schwannomas; melanoma, seminoma, teratocarcinoma, osteosarcoma, xenoderoma pigmentosum, keratoctanthoma, thyroid follicular cancer and Kaposi's sarcoma.

In some embodiments the combination therapy of the present invention is combined with radiation therapy.

In one embodiment, the combination therapy of the present invention is optionally selectively administered to subjects exhibiting a proliferative disorder that involves reduction or loss of function of a tumor suppressor gene product, such as the p53 or Rb gene products. In such embodiments, subjects are screened for reduction or loss of function of a tumor suppressor gene product compared with non-affected tissues or subjects, and only those exhibiting such reduction or loss of function are treated using the combination therapy of the present invention. In one embodiment, the aberrantly proliferating tissue of the subject is screened for the presence and/or activity of p53 or Rb gene products to determine whether the subject is suitable for treatment using the combination therapy of the present invention. In such embodiments, acceptable subjects may have reduced or lost function of p53, Rb or both.

In some embodiments, the first agent is specific for the DNA polymerase alpha relative to another DNA polymerase, e.g. DNA polymerase epsilon, by a factor of 1.5-, 2-, 3-, 4-, 5-, 6-, 7-, 8-, 9-, 10-, 12-, 15-, 20-, 25-, 30-, 40-, 50-, 60-, 70-, 80-, 90-, 100-, 150-, 200-, 300-, 400-, 500-, 700-, 1000-fold or more, as measured by the ratio of IC50s of the agent for DNA polymerase epsilon (encoded by the Polε gene) relative to its IC50 for DNA polymerase alpha (encoded by the Polα gene), as expressed by the formula IC50_{Polε}/IC50_{Polα}.

In some embodiments, the second agent is specific for Chk1 relative to another protein kinase, e.g. CDK2, by a factor of 1.5-, 2-, 3-, 4-, 5-, 6-, 7-, 8-, 9-, 10-, 12-, 15-, 20-, 25-, 30-, 40-, 50-, 60-, 70-, 80-, 90-, 100-, 150-, 200-, 300-, 400-, 500-, 700-, 1000-fold or more, as measured by the ratio of IC50s of the agent for CDK2 relative to its IC50 for Chk1, as expressed by the formula IC50_{CDK2}/IC50_{Chk1}. In some embodiments, the IC50 ratio is 5-fold, 10-fold, or 50-fold.

In some embodiments, first agents include binding compounds directed to DNA polymerase alpha, such as antibodies (e.g. intrabodies) or antigen binding fragments thereof. First agents may also include antisense nucleic acids or siRNA directed to PolA.

In some embodiments, second agents include binding compounds directed to a checkpoint kinase (e.g. Chk1), such as antibodies (e.g. intrabodies) or antigen binding fragments thereof. Second agents may also include antisense nucleic acids or siRNA directed to a gene encoding a checkpoint kinase (e.g. Chk1).

In one embodiment, combination therapy is effected using a pharmaceutical composition comprising an amount of a first agent that inhibits DNA polymerase alpha and an amount of a second agent that inhibits Chk1, wherein the administration of the composition to a subject results in a therapeutic effect. In various embodiments the therapeutic effect is prevention, reduction or elimination of aberrant proliferation, e.g. prevention or a tumor, or slowing of the growth or elimination of a tumor or other cancerous tissue in a subject.

In another aspect, the invention relates to use of inhibitors of DNA polymerase alpha and inhibitors of a checkpoint kinase, e.g. Chk1, in the manufacture of a medicament for the treatment of proliferative disorders.

### BRIEF DESCRIPTION OF THE DRAWINGS

FIG. 1 is a western blot of a gel showing Chk1 S345 phosphorylation following treatment with hydroxyurea (HU), gemcitabine (GEM), Ara-C (Ara) or no treatment (“-”). Chk1 was measured as a loading control.

FIG. 2A is a western blot of a gel showing Chk1 S345 phosphorylation following transfection with a control siRNA to luciferase (Luc), with or without hydroxyurea (+/−HU), as compared with specific siRNA duplexes to DNA polymerase alpha (Polα), epsilon (Polε), or delta (Polδ). Rad17 is included as a loading control.

FIG. 2B and FIG. 2C provide plots of γ-H2A.X phosphorylation and DNA content, as assessed by intracellular staining and FACS analysis, for cells transfected with luciferase siRNA (with and without HU treatment) or with specific siRNA duplexes to DNA polymerase alpha (Polα), epsilon (Polε), or delta (Polδ). The proportion of cells (ranging from 0.3% to 3.2%) in each experiment with DNA damage greater than a specified threshold value is provided. For each experiment, a plot is provided of the DNA content of all of the cells counted. Data represent the average of three independent experiments.

FIG. 2D is a western blot of a gel showing Chk1 S345 phosphorylation following transfections with a control siRNA duplex to luciferase (Luc), or to various combinations of Chk1, DNA polymerase alpha (PolA), epsilon (PolE), or delta (PolD).

FIG. 3 is a western blot of a gel showing Chk1 S345 and RPA32 S33 phosphorylation following transfections of specific siRNA duplexes to luciferase (Luc), or to various combinations of Chk1, DNA polymerase alpha (Polα), epsilon (Polε), or delta (Polδ). Rad17 is included as a loading control.

FIG. 4A is a plot of % γ-H2AX phosphorylation (a measure of double stranded DNA breaks) following transfection of siRNA to luciferase (Luc), with or without hydroxyurea (+/−HU), as compared to various combinations of siRNA to Chk1, DNA polymerase alpha (Polα), epsilon (Polε), and delta (Polδ).

FIG. 4B is a plot of γ-H2AX phosphorylation versus DNA content for cells transfected with siRNA to luciferase (Luc) or DNA polymerase alpha (Polα), with or without a small molecule Chk1 inhibitor (2.5 μM, 2 hours), as described in greater detail below. Untreated and DMSO treated cells serve as controls.

FIG. 5 is a western blot of a gel showing Chk1 S345 phosphorylation following transfection with siRNA to luciferase (Luc) or various combinations of siRNA to Chk1, ATR, ATM and DNA polymerase alpha (Polα). Rad17 is included as a loading control.

FIG. 6 is a plot of % H2AX phosphorylation (a measure of double stranded DNA breaks) following transfection with a control siRNA to luciferase (Luc), as compared with treatment with various combinations of siRNA to Chk1, ATR, ATM and DNA polymerase alpha (Polα).

FIG. 7 is a western blot of a gel showing co-immunoprecipitation of Chk1 and Chk1 S345P with DNA polymerase alpha in immunoprecipitations (IP) using anti-Polα monoclonal antibody SJK-132-20 (Tanaka et al. (1982) J. Biol. Chem. 257:8386) or a monoclonal antibody against SV40 T-antigen (Pab 419, Calbiochem, San Diego, Calif.) as a negative control. Results are shown for cells treated with siRNA to luciferase (Luc), Chk1 or ATR, all with or without hydroxyurea (+/−HU).

FIG. 8 is a western blot of a gel showing co-immunoprecipitation of DNA polymerase alpha (Polα) and Chk1 S345P with Chk1 in immunoprecipitations (IP) using anti-Chk1 monoclonal antibody 58D7. Results are shown for cells treated with hydroxyurea (HU), gemcitabine (Gem) or a combination of gemcitabine and an excess of a peptide (cognate immunogen CNRERLLNKMCGTLPYVAPELLKRREF) (SEQ ID NO: 8) that competes with Chk1 for binding to antibody 58D7, as well as untreated (Unt) cells.

FIG. 9 is a western blot of a gel showing co-immunoprecipitation of Chk1 and Chk1 S345P with DNA polymerase alpha in immunoprecipitations (IP) using anti-Polα monoclonal antibody SJK-132-20 (Tanaka et al. (1982) J. Biol. Chem. 257:8386) as a function of length of treatment with HU (in hours).

FIG. 10 is a western blot of a gel showing Chk S345 and RPA32 S33 phosphorylation following transfection with siRNA to luciferase (Luc), Chk1 or ATR, all with or without hydroxyurea (+/−HU).

### DETAILED DESCRIPTION

Each patent, patent application publication or other publication cited herein (including database entries, such as protein and nucleic acid sequences) is hereby incorporated by reference in its entirety.

### I. Definitions

As used above, and throughout this disclosure, the following terms, unless otherwise indicated, shall be understood to have the following meanings:

“A,” “an,” and “the,” include their corresponding plural references unless the context clearly dictates otherwise.

Unless otherwise indicated, “or” does not exclude “and.” For example, a claim reciting “element A or element B” encompasses embodiments with A only, embodiments with B only, and embodiments with both A and B.

“Subject” or “patient” includes both human and animals.

“Mammal” means humans and other mammalian animals.

“Composition” is intended to encompass a product comprising the specified ingredients in the specified amounts, as well as any product which results, directly or indirectly, from combination of the specified ingredients in the specified amounts.

“Inhibit” or “treat” or “treatment” includes a postponement of development of the symptoms associated with a proliferative disorder and/or a reduction in the severity of such symptoms that will or are expected to develop. Thus, the terms denote that a beneficial result has been conferred on a vertebrate subject with a proliferative disorder, or with the potential to develop such a disorder or symptom.

As used herein, the term “therapeutically effective amount” or “effective amount” refers to an amount of an agent, e.g. an inhibitor of DNA polymerase alpha or Chk1, that when administered alone or in combination with an additional therapeutic agent (depending on the context) to a cell, tissue, or subject is effective to prevent or ameliorate a proliferative disorder. Effective amount also means an amount sufficient to allow or facilitate diagnosis. A “therapeutically effective dose” refers to that amount of the agent sufficient to result in amelioration of symptoms, e.g., treatment, healing, prevention or amelioration of the relevant medical condition, or an increase in rate of treatment, healing, prevention or amelioration of such conditions. An effective amount for a particular patient or veterinary subject may vary depending on factors such as the condition being treated, the overall health of the patient, the method route and dose of administration and the severity of side effects (see, e.g., U.S. Pat. No. 5,888,530 issued to Netti et al.). An effective amount can be the maximal dose or dosing protocol that avoids significant side effects or toxic effects. The effect will result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject (see, e.g., Maynard et al. (1996) A Handbook of SOPs for Good Clinical Practice, Interpharm Press, Boca Raton, Fla.; Dent (2001) Good Laboratory and Good Clinical Practice, Urch Publ., London, UK).

As used herein, a “therapeutic agent” is an agent that either alone, or in combination with another agent or agents, is capable of contributing to a desired therapeutic, ameliorative, inhibitory or preventative effect. Such “therapeutic agents” need not necessarily have any therapeutic efficacy when administered alone. For example, a DNA polymerase alpha inhibitor of the present invention or a Chk1 inhibitor of the present invention may not necessarily have therapeutic utility when used separately, but may nonetheless be therapeutically efficacious when used together in the methods of the present invention. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

“Small molecule” is defined as a molecule with a molecular weight that is less than 10 kD, typically less than 2 kD, often less than 1 kD, preferably less than 0.7 kD, and most preferably less than about 0.5 kD. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins, are described (see, e.g., Casset et al. (2003) Biochem. Biophys. Res. Commun. 307:198-205; Muyldermans (2001) J. Biotechnol. 74:277-302; Li (2000) Nat. Biotechnol. 18:1251-1256; Apostolopoulos et al. (2002) Curr. Med. Chem. 9:411-420; Monfardini et al. (2002) Curr. Pharm. Des. 8:2185-2199; Domingues et al. (1999) Nat. Struct. Biol. 6:652-656; Sato and Sone (2003) Biochem. J. 371:603-608; U.S. Pat. No. 6,326,482 issued to Stewart et al).

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of a combination of therapeutic agents of the present invention to a human or animal subject, a cell, tissue, physiological compartment, or physiological fluid.

Unless otherwise indicated, the extent of “inhibition” or “activation” caused by an agent is determined using assays in which a protein, gene, cell, cell culture or organism is treated with a potential inhibiting or activating agent and the results are compared to control samples without the agent. Control samples, i.e., not treated with agent, are assigned a relative activity value of 100%. “Inhibition” is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. “Activation” is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., one or more indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer et al. (2001) Glia 36:235-243; Stanimirovic and Satoh (2000) Brain Pathol. 10:113-126).

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least 10 times the control.

The terms “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a nonlimiting example, a binding compound that consists essentially of a recited amino acid sequence may also include one or more amino acids, including substitutions of one or more amino acid residues, that do not materially affect the properties of the binding compound.

### Antibody-Related Definitions

As used herein, the term “antibody” refers to any form of antibody or fragment thereof that exhibits the desired biological activity. Thus, it is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

As used herein, the term “antigen binding fragment” or “binding fragment thereof” encompasses a fragment or a derivative of an antibody that still substantially retains the desired biological activity of the full-length antibody, e.g. inhibition of DNA polymerase alpha. Therefore, the term “antibody fragment” refers to a portion of a full-length antibody, generally the antigen binding or variable region thereof. Examples of antibody fragments include Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of its inhibitory activity. Preferably, a binding fragment or derivative retains at least 25%, 50%, 60%, 70%, 80%, 90%, 95%, 99% or 100% (or more) of its biological activity, although any binding fragment with sufficient affinity to exert the desired biological effect will be useful. It is also intended that an antigen binding fragment of an antibody can include conservative amino acid substitutions that do not substantially alter its biologic activity.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624 and Marks et al. (1991) J. Mol. Biol. 222:581, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA 81: 6851-6855).

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

As used herein, the term “single-chain Fv” or “scFv” antibody refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv, see Pluckthun (1994) THE PHARMACOLOGY OF MONOCLONAL ANTIBODIES, vol. 113, Rosenburg and Moore eds. Springer-Verlag, New York, pp. 269-315.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079, which are hereby incorporated by reference in their entireties. In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully at, e.g., EP404097B1; WO 93/11161; and Holliger et al., (1993) Proc. Natl. Acad. Sci. USA 90: 6444-6448. For a review of engineered antibody variants generally see Holliger and Hudson (2005) Nat. Biotechnol. 23:1126-1136.

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will generally comprise the same CDR sequences of the parental rodent antibodies, although certain amino acid substitutions may be included to increase affinity or increase stability of the humanized antibody.

The antibodies of the present invention also include antibodies with modified (or blocked) Fc regions to provide altered effector functions. See, e.g., U.S. Pat. No. 5,624,821; WO 2003/086310; WO 2005/120571; WO 2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

The term “fully human antibody” refers to an antibody that comprises human immunoglobulin protein sequences only. Such fully human antibodies may be produced using transgenic mice, or even other animals. See, e.g., Lonberg (2005) Nature Biotechnol. 23:1117. A fully human antibody may contain murine carbohydrate chains if produced in a mouse, in a mouse cell, or in a hybridoma derived from a mouse cell. Similarly, “mouse antibody” refers to an antibody which comprises mouse immunoglobulin sequences only.

“Binding compound” refers to a molecule, small molecule, macromolecule, polypeptide, antibody or fragment or analogue thereof, or soluble receptor, capable of binding to a target. “Binding compound” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, which is capable of binding to a target. When used with reference to antibodies, the term “binding compound” refers to both antibodies and binding fragments thereof. “Binding” refers to an association of the binding compound with a target where the association results in reduction in the normal Brownian motion of the binding compound, in cases where the binding compound can be dissolved or suspended in solution. “Binding composition” refers to a molecule, e.g. a binding compound, in combination with a stabilizer, excipient, salt, buffer, solvent, or additive, capable of binding to a target.

### II. Combination Therapy with Inhibitors of DNA Polymerase Alpha and Chk1

The invention disclosed herein relates to methods, and compositions, for the treatment of proliferative disorders by specific inhibition of DNA polymerase alpha and Chk1, e.g. using specific inhibitors of DNA polymerase alpha and Chk1.

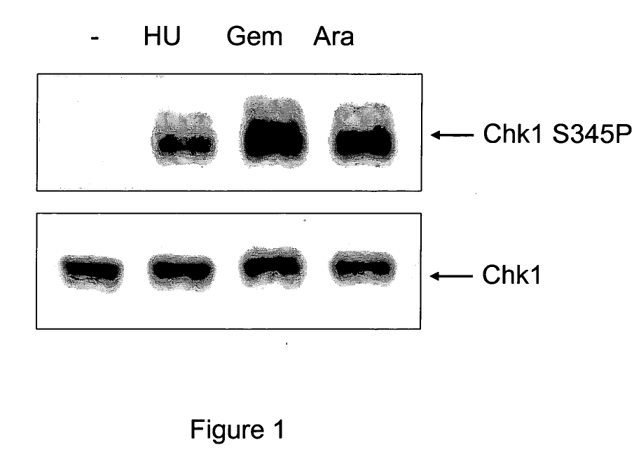

Chk1 is a key effector kinase in cell cycle checkpoint control that becomes activated in response to DNA damage or stalled replication in higher eukaryotes. Liu et al. (2000) Genes Dev. 14:1448; Sorensen et al. (2003) Cancer Cell 3:247; Syljuasen et al. (2005) Mol Cell Biol. 25:3553; Cho et al. (2005) Cell Cycle 4:131. Typically, cells treated with a DNA antimetabolite activate Chk1 as part of the intra-S phase checkpoint to control late origin firing and stabilize stalled replication forks. Feijoo et al. (2001) J. Cell Biol. 154:913; Cho et al. (2005) Cell Cycle 4:131. HU is a ribonucleotide reductase inhibitor that depletes dNTP pools to inhibit DNA replication. Gemcitabine inhibits ribonucleotide reductase, but also blocks DNA replication when incorporated into DNA. Sampath et al. (2003) Oncogene 22:9063. Ara-C is a nucleoside analog that incorporates into DNA and interferes with replicative DNA polymerases. Townsend & Cheng (1987) Mol. Pharmacol. 32:330; Mikita & Beardsley (1988) Biochemistry 27:4698. FIG. 1 confirms that the antimetabolites gemcitabine (Gem), cytarabine (Ara-C, cytosine arabinoside), and hydroxyurea (HU) induce Chk1 S345 phosphorylation, which is a marker of activation of the Chk1 pathway (Liu et al. (2000) Genes Dev. 14:1448; Zhao & Piwnica-Worms (2001) Mol. Cell. Biol. 21:4129; Capasso et al. (2002) J. Cell Sci. 115:4555).

In light of the fact that DNA antimetabolites exert their effects via general suppression of DNA synthesis, it was possible that inhibition of replicative polymerases might provoke activation of the Chk1 pathway. To test this hypothesis, specific siRNA duplexes were used to specifically deplete DNA replication polymerases α, ε, or δ in U20S cells, which were subsequently examined for Chk1 S345 phosphorylation.

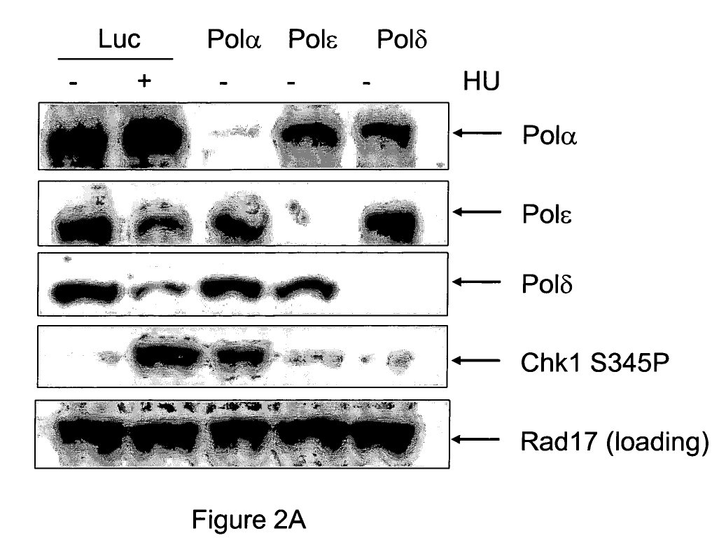

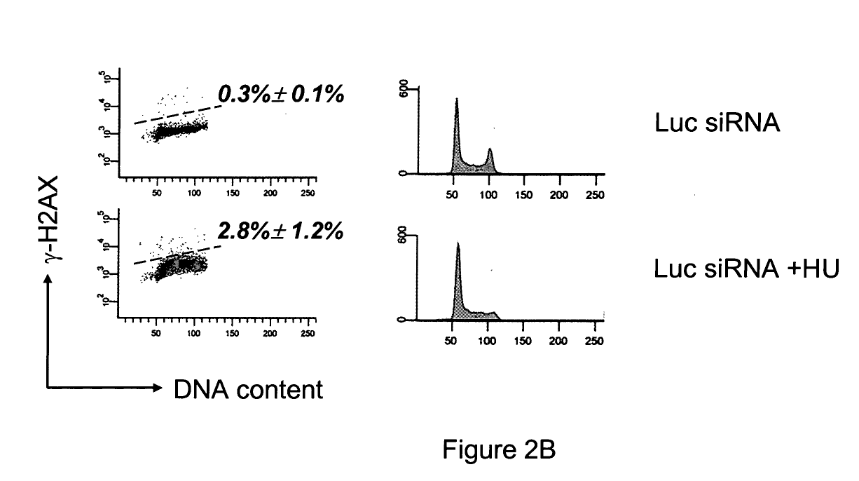

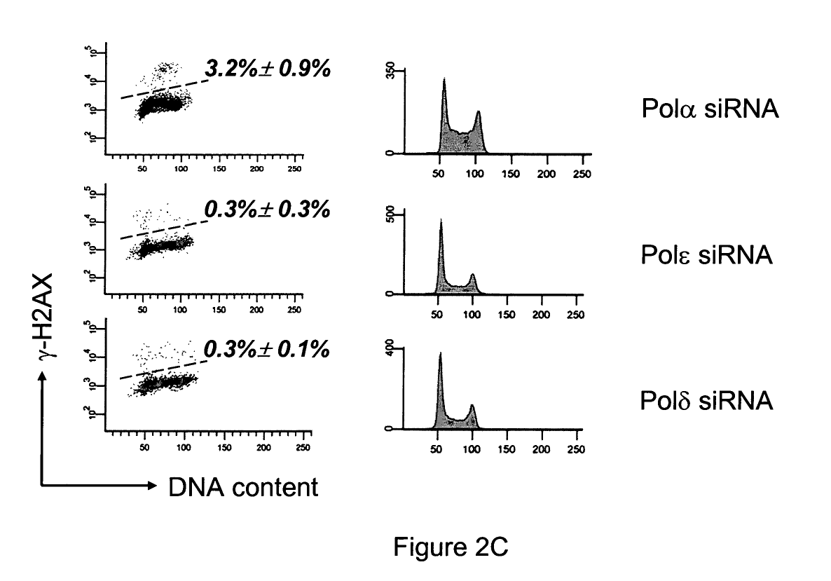

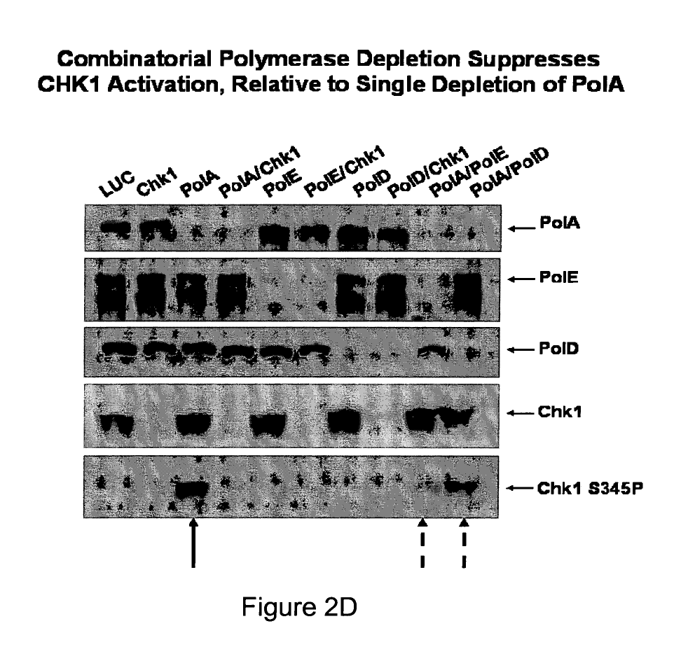

Depletion of Polα by siRNA phenocopies anti-metabolite exposure by inducing Chk1 phosphorylation at residue S345 to generate Chk1 S345P. Specific depletion of Polα induces Chk1 S345 phosphorylation to levels similar to those detectable following HU treatment (FIG. 2A). Depletion of Polε and Polδ did not promote Chk1 S345 phosphorylation under these conditions (FIG. 2A).

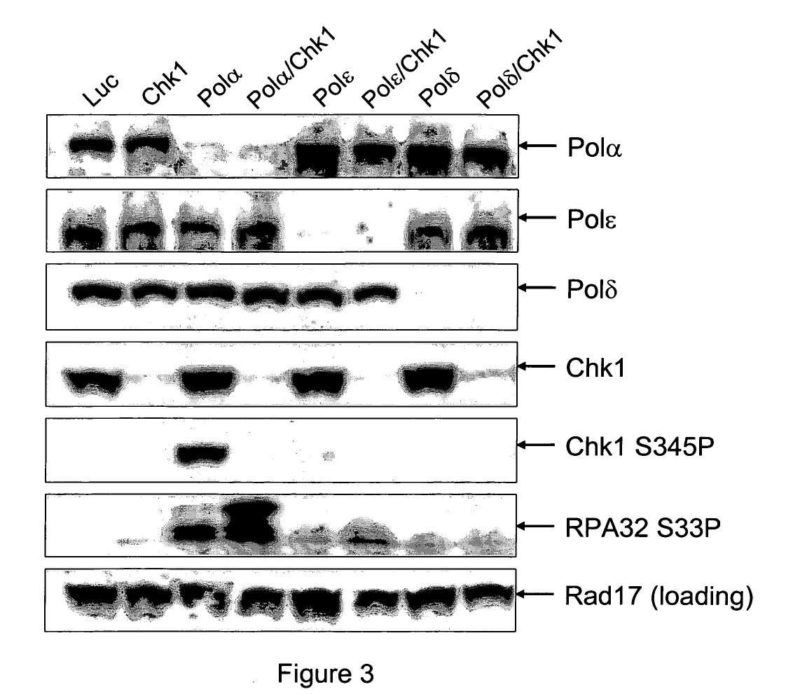

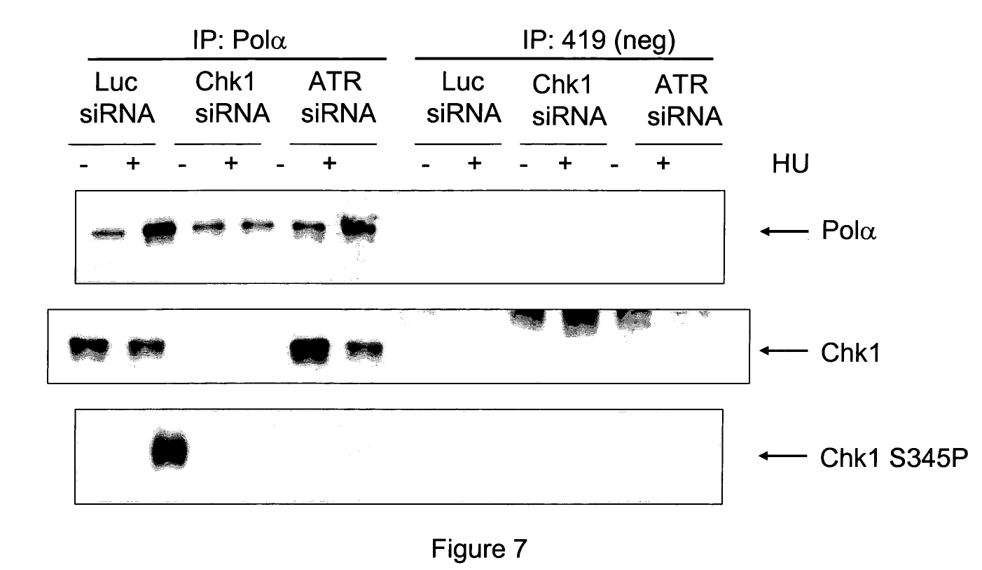

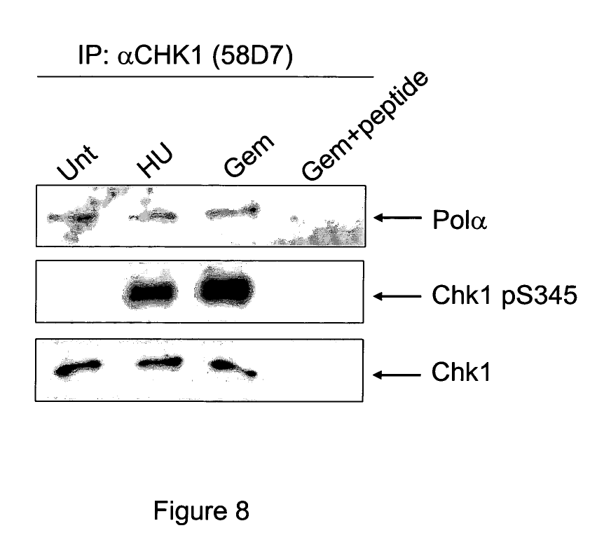

Combinatorial ablation of Polα and Chk1 results in intra-S phase delay and accumulation of DNA damage. See FIGS. 3-6. Consistent with this genetic interaction between Polα and Chk1, Chk1 co-immunoprecipitates with Polα, suggesting a physical interaction. See FIGS. 7-8. Co-depletion of Polα and ATR (and to a lesser extent ATM) yields a similar phenotype, suggesting that ATR and Chk1 are epistatic and required for maintenance of genomic integrity following replication stress. Following replication stress, Polα-associated Chk1 becomes rapidly phosphorylated on S345 in an ATR-dependent manner. Significantly, the ability to efficiently phosphorylate Chk1 in this context is correlated with suppression of DNA damage.

We next examined γ-H2A.X phosphorylation, a marker of double-stranded DNA breaks (Rogakou et al. (1998) J. Biol. Chem. 273:5858; Nazarov et al. (2003) Radiat. Res. 160:309). γ-H2A.X phosphorylation, as assessed by intracellular staining and FACS analysis, was moderately enhanced in Polα depleted cells and expressed preferentially in 3N populations, suggestive of DNA damage within cells traversing S-phase (FIGS. 2B and 2C). In contrast, Polε and Polδ depleted cells showed no accumulation of γ-H2A.X (FIG. 2C). Thus, specific depletion of Polα induced Chk1 S345 phosphorylation and mild intra-S defects.

FIG. 2D demonstrates that ablation of Polα alone induces greater phosphorylation of Chk1 than co-ablation of Polα with Polε, or Polα with Polδ This surprising result suggests that the most desirable DNA polymerase alpha inhibitors for use in the present invention should be highly specific for Polα, and particularly that the inhibitor should exhibit preferential inhibition of Polα over inhibition of Polε. Broad spectrum DNA polymerase inhibitors, such as aphidicolin, would not be suitable for use as DNA polymerase alpha-specific agents in the methods and compositions of the present invention. DNA polymerase alpha-specific inhibitors suitable for use in the methods and compositions of the present invention will preferentially inhibit the activity of Polα relative to Polε by a ratio of 1.5-, 2-, 3-, 4-, 5-, 6-, 7-, 8-, 9-, 10-, 12-, 15-, 20-, 25-, 30-, 40-, 50-, 60-, 70-, 80-, 90-, 100-, 150-, 200-, 300-, 400-, 500-, 700-, 1000-fold or more. This ratio is determined as the ratio of the IC50 of the compound in question, i.e. the concentration needed to achieve half-maximal inhibition, for Polα relative to the IC50 for Polε. The IC50 is determined by a standard DNA polymerase assay as described in Oshige et al. (2004) J. Bioorg. Med. Chem. 12:2597; Mizushina et al. (1997) Biochim. Biophys. Acta 1308:256; Mizushina et al. (1997) Biochim. Biophys. Acta 1336:509. See Example 8.

Genetic studies in fission yeast have suggested a link between Polα and the intra-S checkpoint (Bhaumik & Wang (1998) Mol. Cell Biol. 9:2107). Whilst in Xenopus extracts, DNA synthesis, driven by Polα, is required for full activation of Chk1 following DNA damage (Byun et al. (2005) Genes & Dev. 19:1040).

The results described in Examples 4-7 reveal, for the first time, that Polα genetically, biochemically and functionally interacts with Chk1 in mammalian cells. Moreover, the appropriate regulation of Chk1 activity within this complex, primarily by ATR, is required to suppress DNA damage following replication stress.

These observations further demonstrate that combination of a selective checkpoint activator (i.e. a DNA Polα inhibitor) with a selective Chk1 inhibitor results in a synergistic effect. The expected phenotypes include replication fork collapse, accumulation of DNA damage and onset of apoptosis. The invention relates to compositions and methods to effect this dual inactivation, including use of inhibitors of both Polα and Chk1, e.g. therapeutic agents. Although therapeutic agents, such as drugs, are traditionally used in the treatment of various diseases, any method of inhibiting the activity of Polα and/or Chk1 may be used in the methods of the present invention, even if such inhibition is effected without administration of any therapeutic agent, drug or substance.

### The Role of Polα, Polε, and Polδ in Chk1-Dependent Checkpoint Activation

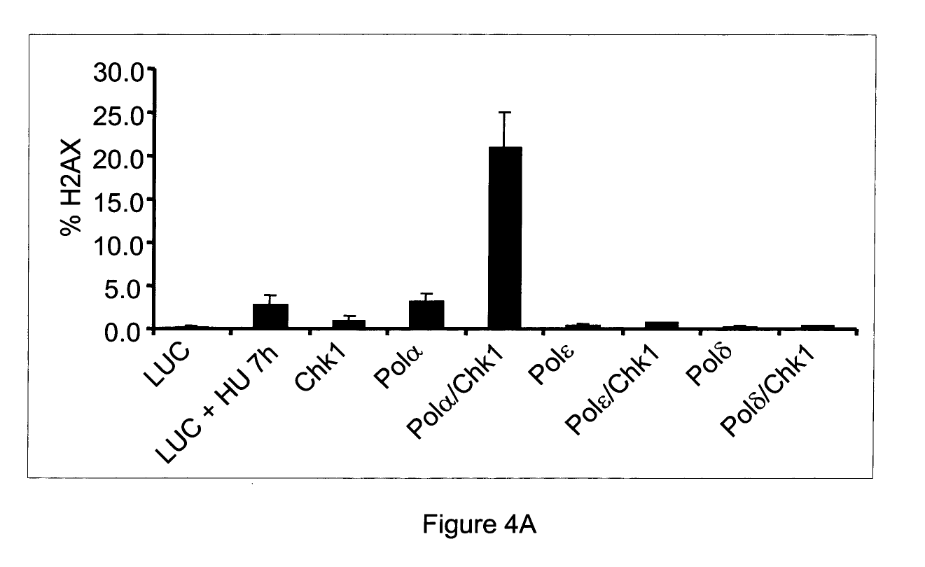

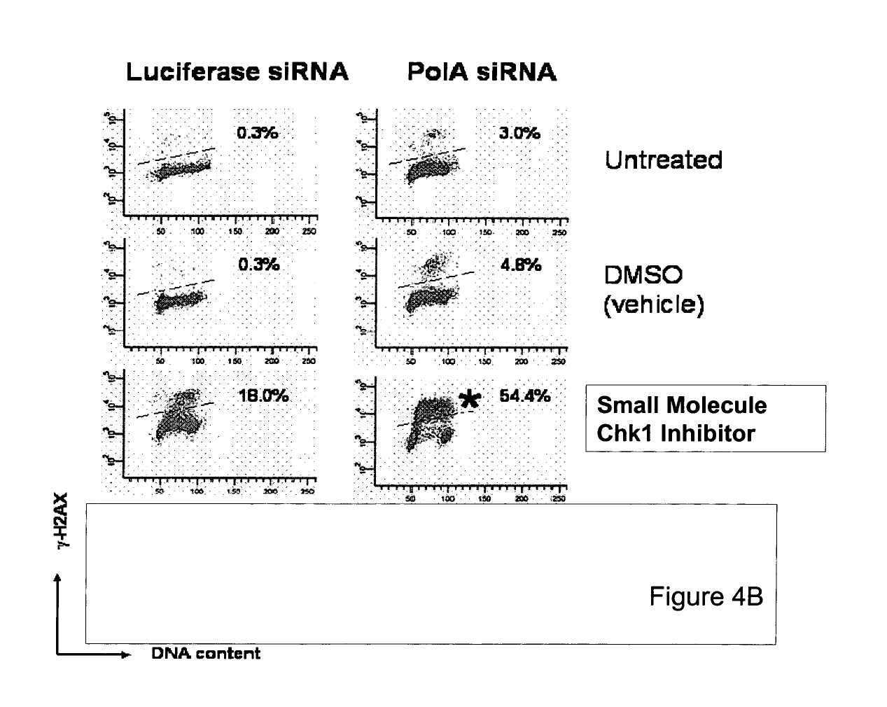

Previous work has shown Chk1 can suppress DNA damage during replication stress (Cho et al. (2005) Cell Cycle 4:131). To test the hypothesis that Chk1 might similarly be required to suppress DNA damage following Polα depletion, we examined DNA damage phenotypes in cells following co-depletion of Polα, Polε, or Polδ with Chk1. Significantly, only co-depletion of Polα and Chk1 triggered RPA32 phosphorylation, in contrast to the Polε/Chk1 and Polδ/Chk1 combinations and the luciferase control (FIG. 3). Similarly, quantitative examination of γ-H2A.X phosphorylation using FACS revealed a strongly-staining population of cells following Polα/Chk1 co-depletion (FIG. 4A). These cells accumulated with an approximately 3N ploidy (as assessed by PI staining), suggestive of specific intra-S phase defects not observed following Polε/Chk1, Polδ/Chk1 or control siRNA depletion (FIG. 4A). Of note, and consistent with prior observations using anti-metabolites (Cho et al. (2005) Cell Cycle 4:131), co-depletion of Polα/Chk1 resulted in significantly enhanced γ-H2A.X phosphorylation compared to cells singly depleted for Polα or Chk1 (FIG. 4A), illustrating the synergistic effect of the combination therapy of the present invention.

FIG. 4B demonstrates that a small molecule inhibitor of Chk1 (3-amino-6-{3-[({[4-(methyloxy)phenyl]methyl}amino)carbonyl]phenyl}-N-[(3S)-piperidin-3-yl]pyrazine-2-carboxamide) is able to generate the same results as ablation of Chk1 with a specific siRNA duplex, in that combination treatment with the small molecule Chk1 inhibitor and siRNA ablation of PolA increases the percentage of cells exhibiting substantial double-stranded DNA breaks from less than 1% in the controls to over 50%.

Thus, specific depletion of Polα (but not Polε or Polδ) induces Chk1 S345 phosphorylation, suggestive of Chk1-dependent checkpoint activation. Similarly, co-depletion of Polα and Chk1 enhanced the accumulation of DNA damage markers (H2A.X S139 and RPA32 S33). These effects were not observed when Polε or Polδ are co-depleted with Chk1, suggesting specificity of the response profile. These data suggest distinct roles for Polα, Polε, and Polδ at the replication fork.

ATR is an upstream activator of Chk1 phosphorylation in response to DNA damage or replication stress (Liu et al. (2000) Genes Dev. 14:1448; Zhao & Piwnica-Worms (2001) Mol. Cell. Biol. 21:4129. Chk1 is phosphorylated on Ser 317 and 345 and activated by ATR in response to stalled replication forks (Liu et al. (2000) Genes Dev. 14:1448; Hekmat-Nejad et al. (2000) Curr. Biol. 10:1565.; Zhao & Piwnica-Worms (2001) Mol. Cell. Biol. 21:4129). We therefore examined the epistatic relationship between Chk1, ATR and ATM following Polα depletion.

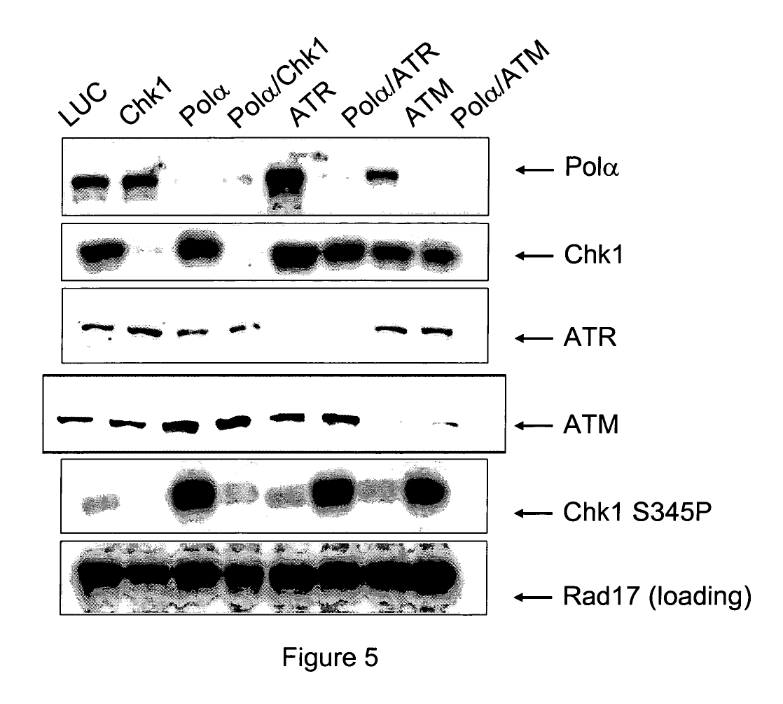

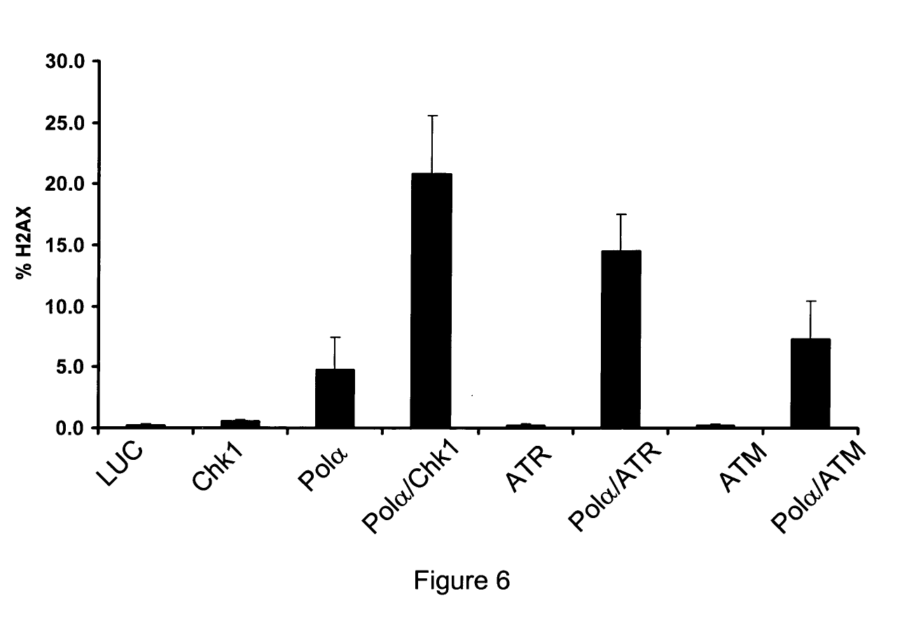

Co-depletion of ATR or ATM with Polα did not alter the accumulation of detectable Chk1 S345 phosphorylation, i.e. phospho-S345 induction was similar to that detected in lysates prepared from cells singly depleted of Polα (FIG. 5), suggesting that in the absence of either ATR or ATM, a cellular pool of Chk1 becomes activated. Single depletion of Polα led to an accumulation of γ-H2A.X in 4.8% of transfected cells, compared to 0.2% for luciferase control siRNA, ATR siRNA, or ATM siRNA, and 0.5% for Chk1 siRNA (FIG. 6). However, combined siRNA knockdown of Polα/Chk1, Polα/ATR, and Polα/ATM yielded γ-H2A.X positive fractions of 21%, 14.6%, and 7.3%, respectively (FIG. 6). Thus, combined depletion of Polα/ATR yielded a phenotype similar to that observed following co-ablation of Polα and Chk1, albeit with somewhat reduced penetrance. Combined depletion of Polα/ATM generated an γ-H2A.X phenotype that was reduced relative to the Polα/ATR combination, the γ-H2A.X signal being slightly elevated relative to that observed following single depletion of Polα.

Overall, these observations suggest that Chk1 activation, driven primarily by ATR, is essential for suppression of DNA damage following depletion of Polα. Whilst specific depletion of either ATR or ATM had little discernable effect on total cellular accumulation of Chk1 S345 following Polα knockdown (FIG. 5), functional suppression of DNA damage during replication stress appears to be mediated primarily via ATR and Chk1, although a contribution from ATM cannot be ruled out.

Physical Interaction of Chk1 S345P with Polα

The strong genetic and functional interactions between Polα, Chk1 and ATR raised the possibility of a direct biochemical interaction between Polα and the intra-S checkpoint apparatus, and specifically Chk1. Polα was immunoprecipitated from U20S cells that had been previously treated with hydroxyurea to induce replication stress. Following SDS-PAGE and western blotting, Polα immune complexes were found to contain readily detectable levels of Chk1 (FIG. 7). The association between Polα and Chk1 did not require hydroxyurea, or ATR. As expected, Chk1 was not detectable in Polα immunoprecipitations prepared from cells depleted of Chk1. Of note, exposure to hydroxyurea led to an accumulation of readily detectable Chk1 S345 within Polα immunoprecipitates, but not in cells depleted of ATR (FIG. 7).

The complementary immunoprecipitation experiment was also run. Immunoprecipitation of Chk1 followed by SDS-PAGE and western blotting demonstrated the presence of endogenous Polα within Chk1 immune complexes (FIG. 8). Again, the interaction with Chk1 appeared constitutive whereas accumulation of the phospho-S345 within Chk1 immune complexes was induced by exposure to HU or GEM. A control experiment was performed in the presence of competing cognate immunogen peptide (SEQ ID NO: 8) for the anti-Chk1 antibody to confirm specificity of the immunoprecipitation (FIG. 8).

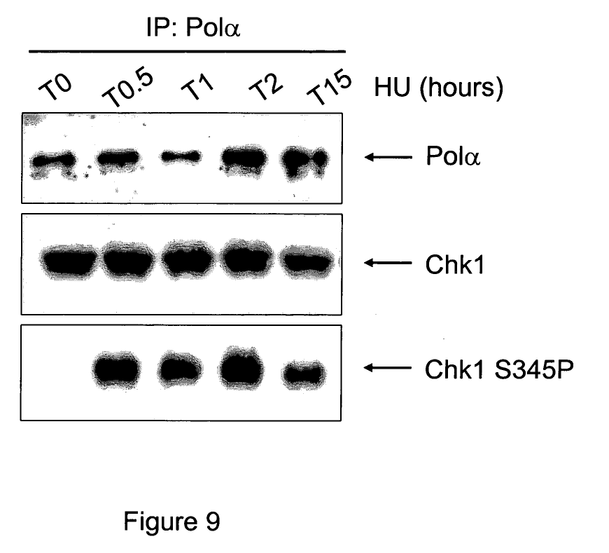

Taken together, these immunoprecipitation results suggest that Chk1 is associated with Polα in proliferating cells, and that the checkpoint effectors are in close proximity to their potential targets. If so, one might expect that this apparently pre-assembled complex should respond rapidly to engagement of the replication checkpoint. A timecourse of Chk1 S345 accumulation in Polα complexes revealed that the Chk1 S345 response was complete at the first time point tested, i.e. 0.5 hours (FIG. 9). Overall, these data suggest that Chk1 associates with Polα, and, in an ATR-dependent manner, can be rapidly phosphorylated within this context following replication stress (hydroxyurea). These observations are in agreement with the functional data suggesting a genetic interaction between Polα, Chk1, and ATR.

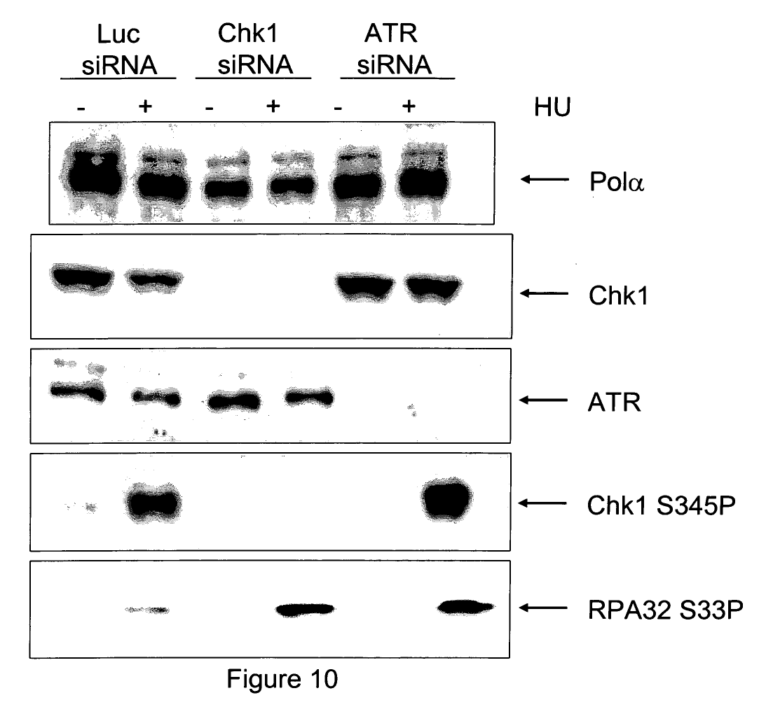

Direct immunoblotting of whole cell extracts confirmed that Chk1 and ATR were depleted following transfection with their respective siRNAs, whereas Polα levels were essentially unaffected by Chk1 or ATR depletion (FIG. 10). In cells transfected with the luciferase (control) siRNA, exposure to HU elicited strong Chk1 S345 phosphorylation and low level RPA32 phosphorylation, consistent with the existence of a functional intra-S checkpoint under these conditions. Levels of Chk1 S345 were also elevated by HU treatment of cells transfected with the ATR siRNA, but RPA32 was phosphorylated at high levels, similar to those observed in HU-treated cells transfected with Chk1 siRNA (FIG. 10). Thus, although HU induces similar levels of Chk1 S345 phosphorylation in the presence or absence of ATR, DNA damage is enhanced when ATR is absent. Overall, these results reveal that HU induces Chk1 S345P in the presence or absence of ATR (FIG. 10) but that Chk1 bound to Polα does not become phosphorylated on S345 (FIG. 7). Because HU-induced DNA damage is suppressed only when ATR is present (FIG. 10), and Chk S345P forms an immunoprecipitable complex with Polα only when ATR is present (FIG. 7), it is possible that the appropriate suppression of DNA damage following replication stress is dependent on the formation of Chk1 S345P-Polα complexes, and that it is these complexes that are responsible for suppression of DNA damage.

### III. DNA Polymerase Alpha Inhibitors

Any method of inhibiting DNA polymerase alpha can be used in the methods of the present invention, and any agent capable of inhibiting DNA polymerase alpha can be used in the compositions of the present invention. DNA polymerase alpha specific inhibitors of the present invention are specific inhibitors of the alpha (α) chain of the eukaryotic DNA polymerase alpha, e.g. as encoded by human PolA, as opposed to other DNA polymerases. Sequence information and other relevant data relating to human DNA polymerase alpha may be found in public databases, such as GenBank Accession numbers NP_{—}058633 and NM_{—}016937, and at Mendelian Inheritance in Man Accession No. 312040, and GeneID No. 5422. Database entries are available on the NCBI Entrez website. This information may be particularly useful in the design and generation of macromolecular inhibitors, such as antisense nucleic acids, siRNA and antibodies.

As used herein, the term “specific” refers to selectivity of binding with respect to the subtype of DNA polymerase, such as DNA polymerase alpha (α), beta (β), epsilon (s) and gamma (γ). In various embodiments the DNA polymerase alpha inhibition is effected using a specific method (or agent) that inhibits DNA polymerase alpha with an IC50 that is 1.5-, 2-, 3-, 4-, 5-, 6-, 7-, 8-, 9-, 10-, 12-, 15-, 20-, 25-, 30-, 40-, 50-, 60-, 70-, 80-, 90-, 100-, 150-, 200-, 300-, 400-, 500-, 700-, 1000-fold or more lower (i.e. more efficacious) than the IC50 for DNA polymerase ε or δ. In other embodiments the DNA polymerase alpha inhibition is effected using a selective method (or agent) that inhibits DNA polymerase α and no more than one other DNA polymerase with IC50s that are 1.5-, 2-, 3-, 4-, 5-, 6-, 7-, 8-, 9-, 10-, 12-, 15-, 20-, 25-, 30-, 40-, 50-, 60-, 70-, 80-, 90-, 100-, 150-, 200-, 300-, 400-, 500-, 700-, 1000-fold or more lower (i.e. more efficacious) than the IC50 for DNA polymerase ε or δ. In some, but not all, embodiments, the DNA polymerase inhibitor preferentially inhibits DNA polymerase α rather than DNA polymerase ε.

In yet further embodiments, the specificity for DNA polymerase alpha as compared with other DNA polymerases is measured by a ratio of affinity measurements other than IC50, such as the Michaelis constant (Km), or the association (Kₐ) or dissociation (IQ) equilibrium binding constant. In each case, the ratio of affinities can range from 1.5-, 2-, 3-, 4-, 5-, 6-, 7-, 8-, 9-, 10-, 12-, 15-, 20-, 25-, 30-, 40-, 50-, 60-, 70-, 80-, 90-, 100-, 150-, 200-, 300-, 400-, 500-, 700-, 1000-fold or more. In yet further embodiments, the ratios of association (kₐ) and dissociation (k_{d}) rate constants may be used. In one embodiment, the rate constant or equilibrium binding constant is determined using surface plasmon resonance spectroscopy, e.g. using a Biacore® instrument (Biacore® Inc., Piscataway, N.J.), in which a DNA polymerase alpha, or an inhibitor of interest, is bound to the surface of a sensor chip, e.g. a sensor chip CM-5 (Biacore® Inc.). This sensor chip is then exposed to the other binding partner to determine the rate or binding constant using standard procedures. See, e.g., Thurmond et al. (2001) Eur. J. Biochem. 268:5747.

Exemplary methods of determining DNA polymerase alpha inhibition activity and specificity are provided herein, and others may be found, e.g., at Togashi et al. (1998) Biochem. Pharmacol. 56:583; Mizushina et al. (2001) Biol. Pharm. Bull. 24:982; Oshige et al. (2004) Bioorganic & Med. Chem. 12:2597; Kamisuki et al. (2004) Bioorganic & Med. Chem. 12:5355; Murakami-Nakai (2004) Biochimica et Biophysica Acta 1674:193; Kamisuki et al. (2002) Biochem. Pharmacol. 63:421; Mizushina et al. (2000) J. Biol. Chem. 275:33957; Mizushina et al. (1997) Biochim. Biophys. Acta 1308:256; Mizushina et al. (1997) Biochim. Biophys. Acta 1336:509. An exemplary method of determining the specificity of an agent for DNA polymerase alpha is provided at Example 8, but other methods known to those skilled in the art may be used.

In some embodiments, specific inhibition of DNA polymerase alpha is effected using small molecules. Exemplary compounds that preferentially inhibit the activity of DNA polymerase alpha include, but are not limited to, 4-hydroxy-17-methylincisterol (Togashi et al. (1998) Biochem. Pharmacol. 56:583), the glycolipid galactosyldiacylglycerol (GDG) (Mizushina et al. (2001) Biol. Pharm. Bull. 24:982), the paclitaxel derivative cephalomannine (Oshige et al. (2004) Bioorganic & Med. Chem. 12:2597), dehydroaltenusin (Kamisuki et al. (2004) Bioorganic & Med. Chem. 12:5355; Murakami-Nakai (2004) Biochimica et Biophysica Acta 1674:193; Kamisuki et al. (2002) Biochem. Pharmacol. 63:421; Mizushina et al. (2000) J. Biol. Chem. 275:33957), 6-(p-n-butylanilino)uracil (CAS 21332-96-7) and N2-(p-butylphenyl)guanine (CAS 83173-14-2) (Rochowska et al. (1982) Biochimica et Biophysica Acta, Gene Structure and Development 699:67).

Exemplary compounds that preferentially inhibit the activity of DNA polymerase alpha and beta include, but are not limited to, sulfolipid compounds (e.g. sulfoquinovosyldiacylglycerol) (Mizushina et al. (1998) Biochem. Pharmacol. 55:537; Ohta et al. (1999) Biol. Pharm. Bull. 22:111) and the paclitaxel metabolite taxinine (Oshige et al. (2004) Bioorganic & Med. Chem. 12:2597).

Exemplary compounds that preferentially inhibit the activity of DNA polymerase alpha and epsilon include, but are not limited to, acyclic phosphonomethoxyalkyl nucleotide analogs, e.g. 9-(2-phosphonomethoxyethyl)guanine diphosphate. Kramata et al. (1996) Mol. Pharmacol. 49:1005.

Exemplary compounds that preferentially inhibit the activity of DNA polymerase alpha, beta and lambda include, but are not limited to, resveratrol (3,4,5-trihydroxystilbene). Locatelli et al. (2005) Biochem. J. 389:259. Resveratrol has been shown to activate Chk1. Tyagi et al. (2005) Carcinogenesis 26:1978.

Other, less specific inhibitors of DNA polymerases, include the triterpene dicarboxylic acid mispyric acid. Mizushina et al. (2005) Biosci. Biotechnol. Biochem. 69:1534.

Certain compounds that were previously believed to specifically inhibit DNA polymerase alpha, e.g. aphidicolin (see, e.g., Haraguchi et al. (1983) Nucl. Acids Res. 11:1197), have been subsequently shown to be less specific than originally believed. See, e.g., Kamisuki et al. (2004) Bioorganic &Med. Chem. 12:5355; Oshige et al. (2004) J. Bioorg. Med. Chem. 12:2597; Popanda et al. (1995) J. Mol. Med. 73:259. Although such compounds cannot be used as the DNA polymerase alpha-specific inhibitors in the methods and compositions of the present invention, they may be used as additional agents (e.g. third agents) in combination with DNA polymerase alpha-specific inhibitors and Chk1 inhibitors of the present invention.

In some embodiments, DNA polymerase alpha inhibitors of the present invention exhibit IC50 values of less than about 5000, 2000, 1000, 500, 250, 100, 50, 25, 10, 5, 2.5, 1, 0.5 nM or 0.1 nM.

Additional compounds that can be used to selectively inhibit DNA polymerase alpha include siRNA (e.g. SEQ ID NO: 3) (see, e.g., Stevenson (2004) New. England. J. Med. 351:1772), antisense RNA, and antibodies, including intrabodies (e.g. Alvarez et al. (2000) Clinical Cancer Research 6:3081). Antibodies to DNA polymerase alpha are disclosed at Tanaka et al. (1982) J. Biol. Chem. 257:8386 and Miller et al. (1985) J. Biol. Chem. 260:134.

In some embodiments, selective DNA polymerase alpha inhibitors are used that are not capable of being incorporated into DNA. Such non-incorporatable inhibitors may cause prolonged arrest of DNA synthesis, enhancing the activation of the checkpoint and creating a greater synergy between the DNA polymerase inhibitor and the checkpoint kinase (e.g. Chk1) inhibitor. This increased synergy may result in enhanced specificity for inducing mitotic crisis preferentially in aberrantly proliferating cells, and thus decreased toxicity when compared with other therapeutic approaches.

### IV. Chk1 Inhibitors

Any means of inhibiting Chk1 can be used in the methods of the present invention, and any agent capable of inhibiting Chk1 can be used in the compositions of the present invention. Sequence information and other relevant data relating to human Chk1 may be found in public databases, such as GenBank Accession numbers NM_{—}001274, AAH04202 and NP_{—}001265, and at Mendelian Inheritance in Man Accession No. 603078, and GeneID No. 1111. All these database entries are available on the NCBI Entrez website. This information may be particularly useful in the design and generation of macromolecular inhibitors, such as antisense nucleic acids, siRNA and antibodies.

In some embodiments the method of inhibiting Chk1 (or agent for inhibiting Chk1) specifically inhibits Chk1 relative to other protein kinases. In various embodiments the Chk1 is inhibited 1.5-, 2-, 3-, 4-, 5-, 6-, 7-, 8-, 9-, 10-, 12-, 15-, 20-, 25-, 30-, 40-, 50-, 60-, 70-, 80-, 90-, 100-, 150-, 200-, 300-, 400-, 500-, 700-, 1000-fold or more than other protein kinases as measured by IC50. In some, but not all, embodiments the other protein kinase is CDK2. In some embodiments, the ratio of IC50 of the agent for Chk1 relative to its IC50 for CDK2 is expressed by the formula IC50_{CDK2}/IC50_{Chk1}. In some embodiments, the IC50 ratio is five-fold, ten-fold, or fifty-fold. See, e.g., U.S. Pat. App. Publication No. 2007/0082900.

In yet further embodiments, the specificity for Chk1 as compared with other protein kinases is measured by the ratio of affinity measurements other than IC50, such as the Michaelis constant (Km), or the association (Kₐ) or dissociation (KO equilibrium binding constant. In each case, the ratio of affinities can range from 1.5-, 2-, 3-, 4-, 5-, 6-, 7-, 8-, 9-, 10-, 12-, 15-, 20-, 25-, 30-, 40-, 50-, 60-, 70-, 80-, 90-, 100-, 150-, 200-, 300-, 400-, 500-, 700-, 1000-fold or more. In yet further embodiments, the ratios of association (kₐ) and dissociation (IQ rate constants may be used. Exemplary methods of determining Chk1 kinase inhibition activity and specificity are provided herein (Examples 2 and 3), and others may be found, e.g., at Lyne et al. (2004) J. Med. Chem. 47:1962. Exemplary methods of determining rate constants and equilibrium binding constants for Chk1 inhibitors include surface plasmon resonance spectroscopy, as discussed supra with respect to DNA polymerase alpha inhibitors.

Compounds that may be useful as Chk1 inhibitors in the methods and compositions of the present invention include imidazopyrazines as disclosed in, e.g., U.S. Pat. No. 6,919,341 and U.S. Pat. App. Publication No. 2005/0009832. Other compounds include those disclosed at WO2005/047290; US2005/095616; WO2005/039393; WO2005/019220; WO2004/072081; WO2005/014599; WO2005/009354; WO2005/005429; WO2005/085252; US2005/009832; US2004/220189; WO2004/074289; WO2004/026877; WO2004/026310; WO2004/022562; WO2003/089434; WO2003/084959; WO2003/051346; US2003/022898; WO2002/060492; WO2002/060386; WO2002/028860; JP (1986)61-057587; U.S. Pat. App. Publication No. 2006/0106023; Burke et al. (2003) J. Biological Chem. 278:1450; and Bondavalli et al. (2002) J. Med. Chem. 45:4875.

Compounds that may be useful as Chk1 inhibitors in the methods and compositions of the present invention also include the pyrazolopyrimidines disclosed in commonly-assigned U.S. patent applications published as U.S. Pat. App. Publication Nos. 2007/0082900; 2007/0083044; 2007/0082901; 2007/0082902; 2006/0128725; 2006/0041131 and 2006/0094706; and U.S. Pat. No. 7,196,092.

Compounds that may be useful as Chk1 inhibitors in the methods and compositions of the present invention also include the imidazopyrazines disclosed in commonly-assigned U.S. patent applications published as U.S. Pat. App. Publication Nos. 2007/0105864; and 2007/0117804; and U.S. patent application Ser. No. 11/758,243.

Compounds that may be useful as Chk1 inhibitors in the methods and compositions of the present invention also include UCN-01 (Mizuno et al. (1995) FEBS Lett. 359:259) and structurally related modified indolcarbazole compounds Gö16976 (Kohn et al. (2003) Cancer Res. 63:31), SB-218078 and staurosporine (Jackson et al. (2000) Cancer Res. 60:566; Zhao et al. (2002) J. Biol. Chem. 277:46609), ICP-1 (Eastman et al. (2002) Mol. Cancer Ther. 1:1067) and CEP-3891 (Syljuasen et al. (2004) Cancer Res. 64:9035; Sorensen et al. (2003) Cancer Cell 3:247). See Tao & Lin (2006) Anti-Cancer Agents Med. Chem. (2006) 6:377.

Compounds that may be useful as Chk1 inhibitors in the methods and compositions of the present invention also include isogranulatimide (Roberge et al. (1998) Cancer Res. 58:5701); debromohymenialdisine (DBH) (Curman et al. (2001) J. Biol. Chem. 276:17914); the pyridopyrimidine derivative PD0166285 (Wang et al. (2001) Cancer Res. 61:8211; Li et al. (2002) Radiat. Res. 157:322); scytonemin (U.S. Pat. App. Pub. No. 2002/0022589; Stevenson et al. (2002) J. Pharmacol. Exper. Ther. 303:858); various diaryl ureas as disclosed in U.S. Pat. App. Publication No. 2004/034038 and PCT publications WO 2002/070494, WO 2003/101444 and WO 2005/072733; A-690002 and A-641397 (Chen et al. (Dec. 15, 2006) Int. J. Cancer 119:2784-2794 (e-published in advance of print 3 Oct. 2006); benzimidazole quinolones such as CHR 124 and CHR 600 (Kesicki et al. (2004) 228^{th} ACS Nat'l Mtg.: MEDI-225; WO 2004/018419; U.S. Pat. App. Publication No. 2005/0256157); tricyclic diazopinoindolones such as PF-00394691 (WO 2004/063198; U.S. Pat. App. Publication No. 2005/0075499); 32 various compounds from the Astra-Zeneca compound library (e.g. those shown at FIG. 5 of Lyne et al. (2004) J. Med. Chem. 47:1962); furanopyrimidines and pyrrolopyrimidines (Foloppe et al. (2005) J. Med. Chem. 48:4332); indolinones (Lin et al. (2006) Bioorg. Med. Chem. Lett. 16:421); substituted pyrazines (WO 2003/093297); compound XL844 (ClinicalTrials.gov Identifier: NCT00234481); pyrimidinylindazolyamines (WO 2005/103036); pyrazolopyrimidines (WO 2004/087707); aminopyrazoles (WO 2005/009435 and WO 2002/0006952); 2-ureidothiophenes (WO 2003/029241; WO 2005/016909); pyrimidines (U.S. Pat. App. Publication No. 2004/0186118); pyrrolopyrimidines (WO 2003/0287243); 3-ureidothiophenes (WO 2003/028731); indenopyazoles (WO 2004/080973); triazlones (WO 2004/081008); dibenzodiazepinones (U.S. Pat. App. Publication No. 2004/254159); macrocyclic ureas (WO 2005/047294); pyrazoloquinoloines (WO 2005/028474); peptides and peptidomimetics, such as CBP501 (WO 2001/021771; WO 2003/059942). See Tao & Lin (2006) Anti-Cancer Agents Med. Chem. (2006) 6:377.

Compounds that may be useful as Chk1 inhibitors in the methods and compositions of the present invention also include those disclosed in WO 2005/047294; U.S. Pat. Nos. 6,797,825, 6,831,175, and 7,056,925; WO 2004/076424; WO 2004/080973; WO 2004/014876; and WO 2003/051838.

Compounds that may be useful as Chk1 inhibitors in the methods and compositions of the present invention also include those disclosed in WO 2004/108136 and WO 2004/087707.

Compounds that may be useful as Chk1 inhibitors in the methods and compositions of the present invention also include those disclosed in WO 2006/048745; U.S. Pat. App. Publication No. 2005/250836; WO 2005/009997; WO 2005/009435; WO 2004/063198; WO 2003/091255; and WO 2003/037886.

Compounds that may be useful as Chk1 inhibitors in the methods and compositions of the present invention also include those disclosed in U.S. Pat. Nos. 7,064,215; U.S. Pat. App. Publication Nos. 2005/261307, 2005/256157; WO 2005/047244; WO 2004/018419; and WO 2003/004488.

Compounds that may be useful as Chk1 inhibitors in the methods and compositions of the present invention also include those disclosed in U.S. Pat. Nos. 7,067,506; U.S. Pat. App. Publication Nos. 2003/0069284; and WO 2005/027907.

In some embodiments, Chk1 inhibitors of the present invention exhibit IC50 values of less than about 5000, 2000, 1000, 500, 250, 100, 50, 25, 10, 5, 2.5, 1, 0.5 nM or 0.1 nM.

Nucleic acid based compounds that can be used to selectively inhibit Chk1 include, but are not limited to, siRNA (e.g. SEQ ID NO: 2), antisense oligonucleotides, and ribozymes, as disclosed at U.S. Pat. Nos. 6,211,164, 6,677,445 and 6,846,921; U.S. Pat. App. Publication Nos. 2004/0097446 and 2005/01533925; and PCT publications WO 2003/070888 and WO 2001/057206.

Antibodies, such as intrabodies (e.g. Alvarez et al. (2000) Clinical Cancer Research 6:3081) may also be used to selectively inhibit Chk1.

### V. siRNA

Methods of producing and using siRNA are disclosed, e.g., at U.S. Pat. Nos. 6,506,559 (WO 99/32619); 6,673,611 (WO 99/054459); 7,078,196 (WO 01/75164); 7,071,311 and PCT publications WO 03/70914; WO 03/70918; WO 03/70966; WO 03/74654; WO 04/14312; WO 04/13280; WO 04/13355; WO 04/58940; WO 04/93788; WO 05/19453; WO 05/44981; WO 03/78097 (U.S. patents are listed with related PCT publications). Exemplary methods of using siRNA in gene silencing and therapeutic treatment are disclosed at PCT publications WO 02/096927 (VEGF and VEGF receptor); WO 03/70742 (telomerase); WO 03/70886 (protein tyrosine phosphatase type IVA (Prl3)); WO 03/70888 (Chk1); WO 03/70895 and WO 05/03350 (Alzheimer's disease); WO 03/70983 (protein kinase C alpha); WO 03/72590 (Map kinases); WO 03/72705 (cyclin D); WO 05/45034 (Parkinson's disease). Exemplary experiments relating to therapeutic uses of siRNA have also been disclosed at Zender et al. (2003) Proc. Nat'l. Acad. Sci. (USA) 100:7797; Paddison et al. (2002) Proc. Nat'l. Acad. Sci. (USA) 99:1443; and Sah (2006) Life Sci. 79:1773. siRNA molecules are also being used in clinical trials, e.g., of chronic myeloid leukemia (CML) (ClinicalTrials.gov Identifier: NCT00257647) and age-related macular degeneration (AMD) (ClinicalTrials.gov Identifier: NCT00363714).

Although the term “siRNA” is used herein to refer to molecules used to induce gene silencing via the RNA interference pathway (Fire et al. (1998) Nature 391:806), such siRNA molecules need not be strictly polyribonucleotides, and may instead contain one or more modifications to the nucleic acid to improve its properties as a therapeutic agent. Such agents are occasionally referred to as “siNA” for short interfering nucleic acids. Although such changes may formally move the molecule outside the definition of a “ribo”nucleotide, such molecules are nonetheless referred to as “siRNA” molecules herein. For example, some siRNA duplexes comprise two 19-25 nt (e.g. 21 nt) strands that pair to form a 17-23 basepair (e.g. 19 base pair) polyribonucleotide duplex with TT (deoxyribonucleotide) 3′ overhangs on each strand. Other variants of nucleic acids used to induce gene silencing via the RNA interference pathway include short hairpin RNAs (“shRNA”), for example as disclosed in U.S. Pat. App. Publication No. 2006/0115453.

Although the sense strand of exemplary siRNA molecules to several genes are provided at SEQ ID NOs: 1-7 (e.g. the sense strand of an siRNA for DNA Polα is provided at SEQ ID NO: 3), other sequences may be used to generate siRNA molecules for use in silencing these genes. The sequence of the opposite strand of the siRNA duplexes is simply the reverse complement of the sense strand, with the caveat that both strands have 2 nucleotide 3′ overhangs. That is, for a sense strand “n” nucleotides long, the opposite strand is the reverse complement of residues 1 to (n−2), with 2 additional nucleotides added at the 3′ end to provide an overhang. Where an siRNA sense strand includes two U residues at the 3′ end, the opposite strand also includes two U residues at the 3′ end. Where an siRNA sense strand includes two dT residues at the 3′ end, the opposite strand also includes two dT residues at the 3′ end.

### VI. Generation of Antibodies

Any suitable method for generating monoclonal antibodies may be used. For example, a recipient may be immunized with the DNA polymerase alpha or Chk1 polypeptides, or an antigenic fragment thereof. Any suitable method of immunization can be used. Such methods can include adjuvants, other immunostimulants, repeated booster immunizations, and the use of one or more immunization routes. The eliciting antigen may be a single epitope, multiple epitopes, or the entire protein alone or in combination with one or more immunogenicity enhancing agents known in the art.

Any suitable method can be used to elicit an antibody with the desired biologic properties to inhibit DNA polymerase alpha or Chk1. It is desirable to prepare monoclonal antibodies (mAbs) from various mammalian hosts, such as mice, rodents, primates, humans, etc. Techniques for preparing such monoclonal antibodies may be found in, e.g., Stites et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Thus, monoclonal antibodies may be obtained by a variety of techniques familiar to researchers skilled in the art. Typically, spleen cells from an animal immunized with a desired antigen are immortalized, commonly by fusion with a myeloma cell. See Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse et al. (1989) Science 246:1275-1281.

Other suitable techniques involve selection of libraries of antibodies in phage or similar vectors. See, e.g., Huse et al. (1989) Science 246:1275; and Ward et al. (1989) Nature 341:544. The polypeptides and antibodies of the present invention may be used with or without modification, including chimeric or humanized antibodies. Also, recombinant immunoglobulins may be produced, see Cabilly U.S. Pat. No. 4,816,567; and Queen et al. (1989) Proc. Nat'l Acad. Sci. USA 86:10029-10033; or made in transgenic mice, see Mendez et al. (1997) Nature Genetics 15:146-156; also see Abgenix and Medarex® technologies.

Also contemplated are chimeric antibodies. As noted above, typical chimeric antibodies comprise a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA 81: 6851-6855).

Bispecific antibodies are also useful in the present methods and compositions. As used herein, the term “bispecific antibody” refers to an antibody, typically a monoclonal antibody, having binding specificities for at least two different antigenic epitopes, e.g., DNA polymerase alpha and Chk1. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al. (1983) Nature 305:537. Alternatively, bispecific antibodies can be prepared using chemical linkage. See, e.g., Brennan et al. (1985) Science 229:81. Bispecific antibodies include bispecific antibody fragments. See, e.g., Hollinger et al. (1993) Proc. Natl. Acad. Sci. U.S.A. 90:6444; Gruber et al. (1994) J. Immunol. 152:5368.

### VII. Pharmaceutical Compositions and Medicaments

To prepare pharmaceutical or sterile compositions (or medicaments) for use in the methods of the present invention, the agent or agents are admixed with a pharmaceutically acceptable carrier or excipient, see, e.g., Remington's Pharmaceutical Sciences and U.S. Pharmacopeia: National Formulary, Mack Publishing Company, Easton, Pa. (1984). Inhibitors of DNA polymerase alpha and inhibitors of protein kinases, such as Chk1 kinase, may be administered as separate agents in separate pharmaceutical compositions, or they may be administered as a mixture in a single pharmaceutical composition. When administered as separate agents, the agents can be administered in any order or sequence. For example, a DNA polymerase alpha inhibitor may be administered before, concurrently with, or after administration of an inhibitor of Chk1. Administration of the two agents can overlap for some portions of the treatment regimen and not for other portions of the treatment regimen. In one embodiment, a DNA polymerase alpha-specific inhibitor is administered prior to, and then concurrently with the administration of a Chk1 inhibitor.

Formulations of therapeutic agents or combinations thereof may be prepared by mixing with physiologically acceptable carriers, excipients, or stabilizers in the form of, e.g., lyophilized powders, slurries, aqueous solutions or suspensions (see, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (eds.) (1993) Pharmaceutical Dosage Forms Parenteral Medications, Marcel Dekker, NY; Lieberman et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition (1990) Mack Publishing Co., Easton, Pa.

Liquid form preparations include solutions, suspensions and emulsions. Examples include water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application and the properties of the specific active compound in question (e.g. the affinity, toxicity or pharmacokinetic profile).

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

A kit according to the present invention can use a kit may comprise a therapeutically effective amount of at least one inhibitor of either DNA polymerase alpha or a checkpoint kinase, e.g. Chk1, or a combination of inhibitors of both, or a pharmaceutically acceptable salt, solvate, ester or prodrug of the agent (or agents) and a pharmaceutically acceptable carrier, vehicle or diluent. The kit may optionally include at least one additional anti-cancer agent, wherein the amounts of the agents result in desired therapeutic effect.

Toxicity and therapeutic efficacy of the therapeutic compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Therapeutic combinations exhibiting high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

The mode of administration of the therapeutic agents of the present invention is not particularly important. Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

Selecting an administration regimen for a therapeutic agent depends on several factors, including the serum or tissue turnover rate of the agent, the level of symptoms, the immunogenicity of the entity, and the accessibility of the target cells in the biological matrix. Preferably, an administration regimen maximizes the amount of therapeutic agent delivered to the patient consistent with an acceptable level of side effects. Accordingly, the amount of agent delivered depends in part on the particular agent and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., reduction in the rate of growth of tumor tissue, or alteration of biomarkers associated with therapeutic efficacy.

Methods for co-administration or treatment with additional therapeutic agents, e.g., a cytokine, steroid, chemotherapeutic agent, antibiotic, or radiation, are well known in the art (see, e.g., Hardman et al. (eds.) (2001) Goodman and Gilman's The Pharmacological Basis of Therapeutics, 10^{th} ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). The pharmaceutical composition of the invention may also contain other immunosuppressive or immunomodulating agents. Any suitable immunosuppressive agent can be employed, including but not limited to anti-inflammatory agents, corticosteroids, cyclosporine, tacrolimus (i.e., FK-506), sirolimus, interferons, soluble cytokine receptors (e.g., sTNRF and sIL-1R), agents that neutralize cytokine activity (e.g., inflixmab, etanercept), mycophenolate mofetil, 15-deoxyspergualin, thalidomide, glatiramer, azathioprine, leflunomide, cyclophosphamide, methotrexate, and the like. The pharmaceutical composition can also be employed with other therapeutic modalities such as phototherapy and radiation.

### VIII. Therapeutic Uses

The methods and compositions disclosed herein can be useful in the therapy of proliferative diseases such as cancer, autoimmune diseases, viral diseases, fungal diseases, neurological/neurodegenerative disorders, arthritis, inflammation, anti-proliferative (e.g., ocular retinopathy), neuronal, alopecia, cardiovascular disease and sepsis. Many of these diseases and disorders are listed in U.S. Pat. No. 6,413,974.

More specifically, the methods and compositions of the present invention can be useful in the treatment of a variety of cancers, including (but not limited to) the following: carcinoma, including that of the bladder, breast, colon, kidney, liver, lung, including small cell lung cancer, non-small cell lung cancer, head and neck, esophagus, gall bladder, ovary, pancreas, stomach, cervix, thyroid, prostate, and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including melanoma, seminoma, teratocarcinoma, osteosarcoma, xenoderoma pigmentosum, keratoctanthoma, thyroid follicular cancer and Kaposi's sarcoma.

The methods of the present invention also may be useful in the treatment of any disease process which features abnormal cellular proliferation, e.g., benign prostate hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, viral disease and fungal infections.

The methods of the present invention may induce or inhibit apoptosis. The apoptotic response is aberrant in a variety of human diseases. The methods and compositions of the present invention can be useful in the treatment of cancer (including but not limited to those types mentioned hereinabove), viral infections (including but not limited to herpesvirus, poxvirus, Epstein-Barr virus, Sindbis virus and adenovirus), prevention of AIDS development in HIV-infected individuals, autoimmune diseases (including but not limited to systemic lupus, erythematosus, autoimmune mediated glomerulonephritis, rheumatoid arthritis, psoriasis, inflammatory bowel disease, and autoimmune diabetes mellitus), neurodegenerative disorders (including but not limited to Alzheimer's disease, AIDS-related dementia, Parkinson's disease, amyotrophic lateral sclerosis, retinitis pigmentosa, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteoporosis and arthritis) aspirin-sensitive rhinosinusitis, cystic fibrosis, multiple sclerosis, kidney diseases and cancer pain.

The methods and compositions of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The methods and compositions of the present invention may also be useful in inhibiting tumor angiogenesis and metastasis.

The invention also relates to use of inhibitors of DNA polymerase alpha and inhibitors of a checkpoint kinase, e.g. Chk1, in the manufacture of a medicament for the treatment of proliferative disorders.

### Dosing

A preferred dosage is about 0.001 to 500 mg/kg of body weight/day of an inhibitor of DNA polymerase alpha or an inhibitor of a checkpoint kinase (e.g. Chk1), or 0.001 to 500 mg/kg of body weight/day of each of the inhibitors. An especially preferred dosage is about 0.01 to 25 mg/kg of body weight/day of one or both of these inhibitors. The inhibitor of DNA polymerase alpha and the inhibitor of a checkpoint kinase (e.g. Chk1) can be present in the same dosage unit or in separate dosage units.

Combination Therapy with Additional Therapeutic Agents

The therapeutic agents of the present invention may also be used in combination (administered together, or sequentially in any order) with one or more of anti-cancer treatments such as radiation therapy, and/or one or more additional anti-cancer agents. In preferred embodiments the one or more additional anti-cancer agents do not inhibit subunits of DNA polymerase other than the alpha subunit. The inhibitor of DNA polymerase alpha, the inhibitor of a checkpoint kinase (e.g. Chk1) and the additional anti-cancer agent(s) can be present in the same dosage unit or in separate dosage units.

In some embodiments, the compositions of the present invention (e.g. comprising a DNA polymerase alpha inhibitor and a Chk1 inhibitor) are co-administered with one or more agents, such as anti-cancer agents, either concurrently or sequentially in any sequence. Non-limiting examples of suitable anti-cancer agents include cytostatic agents, cytotoxic agents (such as for example, but not limited to, DNA interactive agents (such as cisplatin or doxorubicin)); taxanes (e.g. taxotere, taxol); topoisomerase II inhibitors (such as etoposide); topoisomerase I inhibitors (such as irinotecan (or CPT-11), camptostar, or topotecan); tubulin interacting agents (such as paclitaxel, docetaxel or the epothilones); hormonal agents (such as tamoxifen); thymidilate synthase inhibitors (such as 5-fluorouracil); anti-metabolites (such as methoxtrexate); alkylating agents (such as temozolomide (Temodar® from Schering-Plough Corporation, Kenilworth, N.J.), cyclophosphamide); Farnesyl protein transferase inhibitors (such as, Sararsar® (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoehtyl]-1-piperidinecarboxamide, or SCH 66336 from Schering-Plough Corporation, Kenilworth, N.J.), tipifarnib (Zarnestra® or R115777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.); signal transduction inhibitors (such as, Iressa® (from Astra Zeneca Pharmaceuticals, England), Tarceva® (EGFR kinase inhibitors), antibodies to EGFR (e.g., C225), Gleevec® (C-abl kinase inhibitor from Novartis Pharmaceuticals, East Hanover, N.J.); interferons such as, for example, intron (from Schering-Plough Corporation), Peg-Intron® (from Schering-Plough Corporation); hormonal therapy combinations; aromatase combinations; ara-C, adriamycin, cytoxan, Clofarabine (Clolar® from Genzyme Oncology, Cambridge, Mass.), cladribine (Leustat® from Janssen-Cilag Ltd.), aphidicolon, Rituxan® (from Genentech/Biogen Idec), sunitinib (Sutent® from Pfizer), dasatinib (or BMS-354825 from Bristol-Myers Squibb), tezacitabine (from Aventis Pharma), 5 ml 1, fludarabine (from Trigan Oncology Associates), pentostatin (from BC Cancer Agency), triapine (from Vion Pharmaceuticals), didox (from Bioseeker Group), trimidox (from ALS Therapy Development Foundation), amidox, 3-AP (3-aminopyridine-2-carboxaldehyde thiosemicarbazone), MDL-101,731 ((E)-2′-deoxy-2′-(fluoromethylene)cytidine) and gemcitabine.

Other anti-cancer (also known as anti-neoplastic) agents that may be used in combination therapy in the methods and compositions of the present invention include, but are not limited to, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin (Eloxatin®), leucovirin, pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17a-ethinylestradiol, diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, Avastin®, Herceptin® (trastuzumab), Bexxar®, Velcade®, Zevalin®, Trisenox®, Xeloda®, vinorelbine, porfimer, Erbitux®, liposomal, thiotepa, altretamine, melphalan, lerozole, fulvestrant, exemestane, fulvestrant, ifosfomide, Rituxan® (rituximab), C225, Campath®.

If formulated as a fixed dose, such combination products employ the compounds of this invention within the dosage range described herein and the other pharmaceutically active agent or treatment within its dosage range. For example, the CDC2 inhibitor olomucine has been found to act synergistically with known cytotoxic agents in inducing apoptosis (Ongkeko et al. (1995) J. Cell Sci. 108:2897). Inhibitors of DNA polymerase alpha and inhibitors of checkpoint kinases (e.g. Chk1) may also be administered sequentially with known anticancer or cytotoxic agents, e.g. when a combination formulation is inappropriate. The invention is not limited in the sequence of administration; inhibitors of DNA polymerase alpha, inhibitors of checkpoint kinases (e.g. Chk1), and optionally additional anticancer or cytotoxic agent(s), may be administered in any sequence. For example, the cytotoxic activity of the cyclin-dependent kinase inhibitor flavopiridol is affected by the sequence of administration with anticancer agents. Bible & Kaufmann (1997) Cancer Research 57:3375. Such techniques are within the skill of persons skilled in the art as well as attending physicians.

### Patient Selection

Although any subject having a proliferative disorder may be considered for treatment using the methods and compositions of the present invention, subjects particularly suitable for use of the methods and compositions of the present invention may be selected based on the presence or absence of mutations or other functional defects that inhibit the activity of the G1/S replication checkpoint. Examples of such functional defects include absence, reduction or loss of function of the product of tumor suppressor genes p53 and retinoblastoma (Rb). Sequence information and other relevant data relating to human p53 may be found in public databases, such as GenBank Accession numbers NP_{—}000537, and at Mendelian Inheritance in Man Accession No. 191170, and GeneID No. 7157. Sequence information and other relevant data relating to human Rb may be found in public databases, such as GenBank Accession numbers NP_{—}000312, and at Mendelian Inheritance in Man Accession No. 180200, and GeneID No. 5925. Database entries are available on the NCBI Entrez website.

As used herein, “absence” and “reduction” refers to either the physical presence of the tumor suppressor gene product or its activity, although activity will necessarily be lacking in cases where the gene product is not physically present. Loss of function of either (or both) of these genes in a cell can lead to aberrant proliferation, but may also lead to enhanced sensitivity to the methods and compositions of the present invention. Loss of function of a tumor suppressor may be measured by analysis of gene expression at the transcription (RNA) or translational (protein) level, or by binding assays or functional assays. The level of transcription can be measured, e.g., by quantitative amplification of the relevant transcript (e.g. TAQMAN® analysis), Southern or Northern blotting, microarrays, serial analysis of gene expression (SAGE) analysis or any other method known in the art. The level of protein expression can be measured, e.g., by immunoblotting (including Western blotting), immunohistochemistry (IHC), 2-dimensional gel electrophoresis or any other method known in the art. Mutations in tumor suppressor genes may be determined by DNA sequencing, cDNA sequencing, microarray detection, immunoblotting with suitably specific reagents, binding or functional assays or any other method known in the art. Exemplary methods of determining the level of expression or activity of p53 are found at U.S. Pat. Nos. 5,552,283; 6,071,726 and 6,110,671. Exemplary methods of determining the level of expression or activity of Rb are found at U.S. Pat. Nos. 5,578,701; 5,650,287; 5,851,991; 5,998,134 and 6,821,740.

The level of expression or activity of a tumor suppressor gene product in a subject is compared to the “normal” level of expression in a cell or tissue with fully functional tumor suppressor, e.g. non-tumor tissue or tissue from a subject without the proliferative disorder. In various preferred embodiments, the ratio of the normal level of expression or activity to the level in the subject in question is 1.2, 1.5, 2, 3, 4, 5, 10, 12, 15, 20, 25, 30, 40, 50, 75, 100, 200, 500 or 1000 or more. In some embodiments, subjects are selected for treatment with the methods or compositions of the present invention based on the ratio of the normal level of expression or activity to the level of expression or activity in the subject in question, e.g. in the tissue exhibiting aberrant proliferation (e.g. a tumor or other cancerous tissue). The specific ratio selected as the cut-off point is selected to ensure that the tissue in question does in fact have a reduction or loss of tumor suppressor gene product expression or activity sufficient to render the tissue more susceptible to treatment with methods or compositions of the present invention than other tissues in the same subject in order to reduce the risk of unwanted side effects.

The following examples are provided to illustrate embodiments of the present invention, and are not intended to limit the invention. Many modifications and variations of this invention can be made without departing from its spirit and scope, as will be apparent to those skilled in the art.

### EXAMPLES

### Example 1

### General Methods

Standard methods in molecular biology are described (Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausubel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan et al. (2001) Current Protcols in Immunology, Vol. 4, John Wiley, Inc., New York).

Methods for flow cytometry, including fluorescence activated cell sorting (FACS), are available (see, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt et al. (2000) Color Atlas of Histology, Lippincott, Williams, and Wilkins, Phila, Pa.; Louis et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTT® Suite (Informax, Inc., Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690).

Cell lines, drugs, and siRNA treatment materials and methods are as follow. Human U20S osteosarcoma cells are grown in DMEM (Mediatech, Herndon, Va.) supplemented with 10% FBS (JRH BioSciences, St. Louis, Mo.), 200 U/ml penicillin, 200 μg/ml streptomycin, and 300 μg/ml L-Glutamine (Cambrex). HU (Sigma, St. Louis, Mo.) is used at 1 mM for 15 hours.

Sequences for siRNA molecules used herein are provided in Table 1. Sense sequences are provided. Oligonucleotides used as siRNA are obtained from Dharmacon RNA Technologies (Lafayette, Colo.).

Cells are transfected with 50 nM siRNA for Chk1, 100 nM siRNA for Luciferase (Luc), PolA, PolE, PolD1, and ATR duplexes using Lipofectamine 2000 (Invitrogen, Carlsbad, Calif.) according to the manufacturer's protocol.

Flow cytometric analysis, e.g. γ-H2AX detection for DNA damage and BrdU incorporation for cell cycle analysis, is performed as described previously (Cho et al. (2005) Cell Cycle 4:131) and analyzed with a BD LSR II (BD BioSciences, San Jose, Calif.) using FacsDIVA software.

Western blot analysis of siRNA knockdowns is performed as follows. Cell pellets are trypsinized, washed with PBS, and lysed in 2×SDS sample buffer (Invitrogen, Carlsbad, Calif.). Protein extracts are separated by SDS-polyacrylamide gel electrophoresis and transfer to Immobilon®-P membrane (Millipore, Billarica, Mass.). Antibodies used in this study are obtained from Santa Cruz Biotechnology, Inc. (Santa Cruz, Calif.) (Polα, Polε, Polδ, Rad17), Cell Signaling Technology, Inc. (Danvers, Mass.) (pS345-Chk1, pT68-Chk2), Stressgen Bioreagents Corp. (San Diego, Calif.) (Chk1), and Bethyl Laboratories, Inc. (Montgomery, Tex.) (pS33-RPA 32).

Additional antibodies used in the studies described herein were prepared as follows. Monoclonal antibodies (58D7, 16H7) were raised by immunizing BALB/c mice with a peptide (CNRERLLNKMCGTLPYVAPELLKRREF) (SEQ ID NO: 8) spanning the activation loop of human CHK1. Splenocytes were fused to the SP2 myeloma cell line. Reactive hybridomas were identified by ELISA and screened for the ability to immunoprecipitate CHK1.

Immunoprecipitation is performed as follows. Cell pellets are lysed in LT250 buffer (50 mM Tris-HCl pH 7.4, 250 mM NaCl, 5 mM EDTA, 0.1% NP-40, 10% glycerol, 1 mM DTT, 1:100 dilution of phosphatase inhibitor set I and II, and protease inhibitor cocktail set 111 (Calbiochem, San Diego, Calif.). Protein concentrations are determined using the Bio-Rad Protein Assay (Bio-Rad, Hercules, Calif.). For immunoprecipitation, protein lysates (2 mg) are incubated with anti-Polα (SJK 132-20) antibody cross-linked to ImmunoPure Protein G beads for 4 hours at 4° C. Pab419 monoclonal Ab against SV40 T antigen is typically used as a negative control.

Additional methods may be found at Cho et al. (2005) Cell Cycle 4:131.

### Example 2

### Chk1 Kinase Assay

An in vitro scintillation proximity assay (SPA) is described that uses recombinant His-CHK1 expressed in the baculovirus expression system as an enzyme source and a biotinylated peptide based on CDC25C as substrate.

### Materials and Reagents:

1) CDC25C Ser 216 (underlined) C-terminally biotinylated peptide substrate (25 mg) stored at −20° C., custom synthesized by Research Genetics: RSGLYRSPSMPENLNRPR-biotin (SEQ ID NO: 9), 2595.4 MW. Full sequence information relating to CDC25C can be found at NP_{—}001781, and at Mendelian Inheritance in Man Accession No. 157680, and GeneID No. 995. These database entries are available on the NCBI Entrez website.

2) His-CHK1, 235 μg/mL, stored at −80° C.

3) D-PBS (without CaCl₂ and MgCl₂): GIBCO Cat. #14190-144.

4) SPA beads: Amersham (Piscataway, N.J.) Cat. # SPQ0032: 500 mg/vial. Add 10 mls of D-PBS to 500 mg of SPA beads to make a working concentration of 50 mg/ml. Store at 40° C. Use within 2 week after hydration.

5) 96-Well White Microplate with Bonded GF/B filter: Packard Bioscience/Perkin Elmer (Wellesley, Mass.) Cat. #6005177.

6) Top seal-A 96 well Adhesive Film: Perkin Elmer (Wellesley, Mass.) Cat. #6005185.

7) 96-well Non-Binding White Polystyrene Plate: Corning (Acton, Mass.) Cat. #6005177.

8) MgCl₂: Sigma (St. Louis, Mo.) Cat. # M-8266.

9) DTT: Promega (Madison, Wis.) Cat. # V3155.

10) ATP, stored at 4° C.: Sigma Cat. # A-5394.

11) γ³³P-ATP, 1000-3000 Ci/mMol: Amersham Cat. # AH9968.

12) NaCl: Fisher Scientific Cat. # BP358-212.

13) H₃PO₄ 85% Fisher Scientific Cat. #A242-500.

14) Tris-HCl pH 8.0: Bio-Whittaker/Cambrex (Baltimore, Md.) Cat. #16-015V.

15) Staurosporine, 100 μg: CALBIOCHEM (San Diego, Calif.) Cat. #569397.

16) Hypure Cell Culture Grade Water, 500 mL: HyClone (Logan, Utah) Cat. # SH30529.02.

### Reaction Mixtures:

1) Kinase Buffer: 50 mM Tris pH 8.0; 10 mM MgCl₂; 1 mM DTT

2) His-CHK1, MW ˜30 kDa, stored at −80° C. 6 nM is required to yield positive controls of ˜5,000 CPM. For 1 plate (100 r×n): dilute 8 μL of 235 μg/mL (7.83 μM) stock in 2 mL Kinase Buffer. This makes a 31 nM mixture. Add 20 μL/well. This makes a final reaction concentration of 6 nM.

3) CDC25C Biotinylated peptide. Dilute CDC25C to 1 mg/mL (385 μM) stock and store at −20° C. For 1 plate (100 r×n): dilute 10 μL of 1 mg/mL peptide stock in 2 ml Kinase Buffer. This gives a 1.925 μM mix. Add 20 μL/r×n. This makes a final reaction concentration of 385 nM.

4) ATP Mix. For 1 plate (100 r×n): dilute 10 μL of 1 mM ATP (cold) stock and 2 μL fresh ³³P-ATP (20 μCi) in 5 ml Kinase Buffer. This gives a 2 μM ATP (cold) solution; add 50 μl/well to start the reaction. Final volume is 100 μl/r×n so the final reaction concentrations will be 1 μM ATP (cold) and 0.2 μCi/r×n.

5) Stop Solution: Prepare a mixture of 10 mL Wash Buffer 2 (2M NaCl 1% H₃PO₄) and 1 mL SPA bead slurry (50 mg) per plate (100 r×n). Add 100 μL/well.

6) Wash Buffer 1: 2 M NaCl.

7) Wash Buffer 2: 2 M NaCl, 1% H₃PO₄.

### Assay Procedure:

| | | |
|---|---|---|
| Assay | Final | |
| Component | Concentration | Volume |

| CHK1 | 6 | nM | 20 μl/rxn |
|---|---|---|---|
| Compound | — | | 10 μl/rxn |
| (10% DMSO) | | | |
| CDC25C | 0.385 | μM | 20 μl/rxn |
| γ³³P-ATP | 0.2 | μCi/rxn | 50 μl/rxn |
| Cold ATP | 1 | μM | |
| Stop solution | 0.5 | mg/rxn | 100 μl/rxn* |
| SPA beads | | | |
| | | | 200 μl/rxn** |

*Total reaction volume for assay.
**Final reaction volume at termination of reaction (after addition of stop solution).

1) Dilute compounds to desired concentrations in water/10% DMSO—this will give a final DMSO concentration of 1% in the r×n. Dispense 10 μl/r×n to appropriate wells. Add 10 μL 10% DMSO to positive (CHK1+CDC25C+ATP) and negative (CHK1+ATP only) control wells.

2) Thaw enzyme on ice—dilute enzyme to proper concentration in kinase buffer (see Reaction Mixtures) and dispense 20 μl to each well.

3) Thaw the biotinylated substrate on ice and dilute in kinase buffer (see Reaction Mixtures). Add 20 μL/well except to negative control wells. Instead, add 204 Kinase Buffer to these wells.

4) Dilute ATP (cold) and ³³P-ATP in kinase buffer (see Reaction Mixtures). Add 50 μL/well to start the reaction.

5) Allow the reaction to run for 2 hours at room temperature.

6) Stop reaction by adding 100 μL of the SPA beads/stop solution (see Reaction Mixtures) and incubate 15 minutes prior to harvest.

7) Place a blank Packard GF/B filter plate into the vacuum filter device (Packard plate harvester) and aspirate 200 mL water through to wet the system.

8) Take out the blank and put in the Packard GF/B filter plate.

9) Aspirate the reaction through the filter plate.

10) Wash: 200 ml each wash; 1× with 2M NaCl; 1× with 2M NaCl/1% H₃PO₄.

11) Allow filter plate to dry 15 min.

12) Put TopSeal-A adhesive on top of filter plate.

13) Run filter plate in Top Count microplate scintillation counter
Settings: Data mode: CPM
   Radionuclide: Manual SPA: ³³P
   Scintillator: Liq/plast
   Energy Range: Low

### IC50 Determinations:

Dose-response curves are plotted from inhibition data generated, each in duplicate, from eight point serial dilutions of inhibitory compounds. Concentration of compound is plotted against percent kinase activity, calculated by CPM of treated samples divided by CPM of untreated samples. To generate IC50 values, the dose-response curves are then fitted to a standard sigmoidal curve and IC50 values are derived by nonlinear regression analysis.

### Example 3

### CDK2 Assay

An in vitro scintillation proximity assay (SPA) is described that uses recombinant cyclin E and CDK2. See U.S. Pat. No. 7,038,045; U.S. Patent App. Publication No. 2006/0030555. Cyclin E (GenBank Accession No. NP_{—}001229) is cloned into pVL1393 (Pharmingen, La Jolla, Calif.) by PCR, with the addition of five histidine residues at the amino-terminal end to allow purification on nickel resin. The expressed protein is approximately 45 kDa. CDK2 (GenBank Accession No. CCA43807) is cloned into pVL1393 by PCR, with the addition of a hemagglutinin epitope tag at the carboxy-terminal end (YDVPDYAS) (SEQ ID NO: 10). The expressed protein is approximately 34 kDa in size.

Recombinant baculoviruses expressing cyclin E and CDK2 are co-infected into SF9 cells at an equal multiplicity of infection (MOI=5) for 48 hrs. Cells are harvested by centrifugation at 1000 RPM for 10 minutes, then pellets are lysed on ice for 30 minutes in five times the pellet volume of lysis buffer containing 50 mM Tris pH 8.0, 150 mM NaCl, 1% NP40, 1 mM DTT and protease inhibitors (Roche Diagnostics GmbH, Mannheim, Germany). Lysates are spun down at 15000 RPM for 10 minutes and the supernatant retained. 5 ml of nickel beads (for one liter of SF9 cells) are washed three times in lysis buffer (Qiagen GmbH, Germany). Imidazole is added to the baculovirus supernatant to a final concentration of 20 mM, then incubated with the nickel beads for 45 minutes at 4° C. Proteins are eluted with lysis buffer containing 250 mM imidazole. Eluate is dialyzed overnight in 2 liters of kinase buffer containing 50 mM Tris pH 8.0, 1 mM DTT, 10 mM MgCl₂, 100 μM sodium orthovanadate and 20% glycerol. Enzyme is stored in aliquots at −70° C.

Cyclin E/CDK2 kinase assays are performed in low protein binding 96-well plates (Corning Inc, Corning, N.Y.). Enzyme is diluted to a final concentration of 50 μg/ml in kinase buffer containing 50 mM Tris pH 8.0, 10 mM MgCl₂, 1 mM DTT, and 0.1 mM sodium orthovanadate. The substrate used in these reactions is a biotinylated peptide derived from Histone H1 (from Amersham, UK). The substrate is thawed on ice and diluted to 2 μM in kinase buffer. Compounds are diluted in 10% DMSO to desirable concentrations. For each kinase reaction, 20 μl of the 50 μg/ml enzyme solution (1 μg of enzyme) and 20 μl of the 2 μM substrate solution are mixed, then combined with 10 μl of diluted compound in each well for testing. The kinase reaction is started by addition of 50 of 2 μM ATP and 0.1 μCi of ³³P-ATP (from Amersham, UK). The reaction is allowed to run for 1 hour at room temperature. The reaction is stopped by adding 200 μl of stop buffer containing 0.1% Triton X-100, 1 mM ATP, 5 mM EDTA, and 5 mg/ml streptavidin coated SPA beads (from Amersham, UK) for 15 minutes. The SPA beads are then captured onto a 96-well GF/B filter plate (Packard/Perkin Elmer Life Sciences) using a Filtermate universal harvester (Packard/Perkin Elmer Life Sciences.). Non-specific signals are eliminated by washing the beads twice with 2M NaCl then twice with 2 M NaCl with 1% phosphoric acid. The radioactive signal is then measured using a TopCount® 96 well liquid scintillation counter (from Packard/Perkin Elmer Life Sciences).

IC50 values are determined as follows. Dose-response curves are plotted from inhibition data generated, each in duplicate, from eight point serial dilutions of inhibitory compounds. Concentration of compound is plotted against percent kinase activity, calculated by CPM of treated samples divided by CPM of untreated samples. To generate IC50 values, the dose-response curves are then fitted to a standard sigmoidal curve and IC50 values are derived by nonlinear regression analysis.

### Example 4

### Depletion of Polα Induces Chk1 S345 Phosphorylation in the Absence of DNA Damage

FIG. 1 demonstrates that antimetabolites induce Chk1 phosphorylation. U20S cells were untreated (“-”) or treated with 1 mM HU, 5 μM Gem, or 5 μM Ara-C for 2 h. Cell extracts were prepared and immunoblotted with a phospho-Chk1 S345 antibody to show phosphorylated Chk1 (Chk1 S345) and Chk1 (loading control). All three antimetabolites induced substantial phosphorylation of Chk1, which is an indicator of Chk1 activation. Liu et al. (2000) Genes Dev. 14:1448; Zhao & Piwnica-Worms (2001) Mol. Cell. Biol. 21:4129; Capasso et al. (2002) J. Cell Sci. 115:4555.

FIG. 2A demonstrates that depletion Polα with siRNA induces Chk1 phosphorlyation, similar to that induced by HU treatment, but that depletion of Polε and Polδ do not substantially induce Chk1 phosphorylation. At 48 h after siRNA transfections, extracts were prepared and immunoblotted with the indicated antibodies. HU-treated cells were treated with 1 mM HU for 7 h before harvest.

FIGS. 2B and 2C provide flow cytometry results for the samples like those shown in FIG. 2A. Gamma-H2A.X phosphorylation levels and DNA content were measured for cells treated with siRNA to luciferase (Luc), with and without HU, or siRNA to DNA polymerase alpha (Polα), epsilon (Polε), and delta (Polδ). Cultures treated with siRNA to DNA polymerase alpha (Polα) and cultures treated with HU (and the control siRNA) contained approximately 10-fold more cells exhibiting DNA damage compared with control cultures and cultures treated with siRNA to other DNA polymerases. Plots are also provided showing cell counts as a function of DNA content, which demonstrate that cultures treated with siRNA to DNA polymerase alpha (Polα) have an increased proportion of cells in mid-S-phase (˜3N, i.e. ˜75 on the DNA Content axis in FIGS. 2B and 2C), and HU treated cultures have a decreased proportion of 4N cells. These results demonstrate that siRNA to DNA polymerase alpha alone, but not the other DNA polymerases tested, induces DNA damage similar to that induced by HU treatment.

FIG. 2D shows results of experiments similar to those of FIG. 2A except that FIG. 2D includes results for co-ablation of combinations of Polα, Polε and Polδ. As was the case in FIG. 2A, ablation of Polα induces Chk1 phosphorylation while ablation of Polε and Polδ do not, but surprisingly co-ablation of Polα/Polε (and perhaps Polα/Polδ) does not induce Chk1 S345P formation to the same extent as ablation of Polα alone. Specifically, the level of Chk1 S345P is much lower in the co-ablation of Polα/Polε lane than in the ablation of Polα lane, while the level of Chk1 (non-phosphorylated) is unchanged.

### Example 5

### Co-Depletion of Polα and Chk1 Induces Intra-S Phase Arrest

Cells were tested for Chk1 and RPA32 phosphorylation as a function of depletion of DNA polymerases alone and in combination with depletion of Chk1. At time 0, cells were transfected with PolA, PolE, or PolD specific duplexes for 24 h followed by Chk1 specific duplexes for 24 h. At 48 h, extracts were prepared and immunoblotted with the indicated antibodies. FIG. 3 shows that Chk1 and RPA32 are phosphorylated in cells treated with siRNA to DNA polymerase alpha, and that RPA32 phosphorylation in significantly increased when cells are treated with siRNA to both Chk1 and DNA polymerase alpha. Data shown represent the average of three independent experiments.

FIG. 4A shows flow cytometry results measuring the level of phosphorylation of H2AX (a measure of double stranded DNA breaks) for the samples like those used to obtain the data in FIG. 3. The results are the average of three independent experiments and error bars represent standard deviations. While HU and Polα siRNA modestly increase H2A.X phosphorylation compared to control samples, the combination of siRNAs to Polα and Chk1 significantly increase H2A.X phosphorylation, demonstrating a synergy of the two agents in the induction of double stranded DNA breaks.

FIG. 4B demonstrates that a small molecule inhibitor of Chk1 (3-amino-6-{3-[({[4-(methyloxy)phenyl]methyl}amino)carbonyl]phenyl}-N-[(3S)-piperidin-3-yl]pyrazine-2-carboxamide) has the same effect as the siRNA Chk1 knockdown when used in combination with siRNA directed to DNA polymerase alpha. As in FIG. 4A, the combination of inhibiting both Chk1 and DNA polymerase alpha leads to a substantial increase in the fraction of cells with significant DNA damage. Collectively, the results in FIGS. 4A and 4B demonstrate a greater-than-additive effect of the combination therapy of the present invention.

### Example 6

### ATR and to a Lesser Extent ATM are Required for Polα-Mediated Intra-S Phase Arrest

ATM and ATR were depleted, either alone or in combination with depletion of Polα, to determine their role in Polα-mediated cell cycle arrest. FIG. 5 shows the results. At time 0, cells that were transfected with two siRNAs (Polα/Chk1, Polα/ATR and Polα/ATM) were transfected with specific duplexes of PolA for 24 h, followed by specific duplexes of Chk1, ATR, or ATM for 24 h. Other samples were transfected with the indicated siRNA for 24 h. At 48 h, extracts were prepared and immunoblotted with the indicated antibodies. Depletion of ATR or ATM alone did not induce Chk1 phosphorylation, and co-depletion of ATM and ATR with Polα did not increase Chk1 phosphorylation compared with depletion of Polα alone.

FIG. 6 is a plot of DNA damage (as measured by H2AX phosphorylation) for the samples like those described with reference to FIG. 5. As shown in FIG. 4, co-depletion of Polα and Chk1 results in a substantial increase in H2AX phosphorylation. Co-depletion of Polα with ATR and ATM also increased H2AX phosphorylation, although to a lesser extent than co-depletion with Chk1. The results are the average of three-six independent experiments and error bars represent standard deviations.

### Example 7

### Physical Association of Polα and Chk1

Immunoprecipitation experiments were performed to determine whether, and under what circumstances, Polα and Chk1 polypeptides form a complex. Cells were transfected with siRNA for luciferase (control), Chk1 or ATR. After 24 h cells were then either treated or not treated with 1 mM HU for 15 h. Polα was immunoprecipitated from luciferase (positive control), Chk1 (negative control), and ATR depleted cells with Polα antibodies (SJK132-20) cross-linked to protein G, and also with a control unrelated antibody (419). Western blots were performed using with anti-Polα, anti-Chk1, and anti-Chk1 S345 antibodies (FIG. 7). Chk1 co-immunoprecipitated with Polα, suggesting that they exist in a complex in solution.

A reciprocal experiment was performed to confirm the association, in which Chk1 was immunoprecipitated from lysates prepared from untreated U20S cells, or cells treated with HU, gemcitabine, or gemcitabine plus a peptide that blocks binding of the anti-Chk1 antibody to Chk1. Following SDS-PAGE, western blots were probed sequentially with antisera specific for Polα, Chk1 S345P and total Chk1 (FIG. 8). Polα co-immunoprecipitated with Chk1 in lysates from untreated and treated cells.

A time course of HU induction of Chk1 phosphorylation was performed. U20S cells were treated with HU for 0.5, 1, 2 and 15 h, and protein extracts were prepared for immunoprecipitation. Polα was immunoprecipitated with Polα antibodies (SJK132-20) cross-linked to protein G. Western blots were performed using anti-Polα, anti-Chk1, and anti-Chk1 S345 antibodies (FIG. 9). Chk1 phosphorylation was complete at even the earliest timepoint, and both Chk1 and Chk1 S345P co-immunoprecipitated with Polα.

FIG. 10 shows whole cell extracts that were subjected to Western blots with anti-Polα, anti-ATR, anti-Chk1, anti-Chk1 S345P, and anti-RPA32 S33 antibodies.

### Example 8

### DNA Polymerase Alpha Specificity

The specificity of inhibition of DNA polymerase alpha, as compared with other DNA polymerases, may be determined by comparing inhibition of DNA polymerase alpha with the inhibition of other DNA polymerases under similar conditions. In the case of an inhibitory agent, the agent may be titrated in a DNA polymerase assay to determine the concentration necessary to achieve a specified level of inhibition, e.g. 50% (the IC50).

An exemplary assay for determining inhibition of a DNA polymerase is by measurement of the incorporation of radioactive nucleotides. See, e.g., Mizushina et al. (1997) Biochim. Biophys. Acta 1308:256; Mizushina et al. (1997) Biochim. Biophys. Acta 1336:509. Inhibition of DNA polymerase alpha may be compared to the inhibition of DNA polymerase epsilon as follows.

Mammalian DNA polymerases alpha and epsilon are prepared from calf thymus by conventional methods. See, e.g., Podust et al. (1992) Chromosoma 102:S133; Focher et al. (1989) Nucleic Acids Res. 17:1805.

A standard mixture is prepared for each polymerase containing 50 mM Tris HCl, pH 7.5, 1 mM dithiothreitol, 1 mM MgCl₂, 5 μM poly(dA)/oligo(dT)₁₂₋₁₈ (=2/1), 10 mM [³H]dTTP (100 cpm/pmol), 15% (v/v) glycerol and 0.05 units of DNA polymerase. One unit of polymerase activity is defined as the amount that catalyses the incorporation of 1 nmol of deoxyribonucleoside triphosphate into synthetic template-primers (i.e., poly(dA)/oligo(dT)₁₂₋₁₈, A/T=2/1) in 60 min at 37° C. under the normal reaction conditions. Twenty-four μl of this polymerase mixture is mixed with 8 μl of a solution of a (putative) polymerase inhibitor solution comprising a buffer or solvent appropriate to solubilize the inhibitor. A series of samples containing different concentrations of inhibitor, empirically determined for each inhibitor, are used to determine the concentration required to inhibit polymerase activity to 50% of the uninhibited level (the IC50). A control sample comprising 8 μl of the buffer or solvent in place of the inhibitor is used to ensure that the buffer and/or solvent do not block the activity of the DNA polymerase in the reaction mixture.

After incubation at 37° C. for 60 min, the radioactive DNA product is collected on a DEAE-cellulose paper disc (DE81) as described by Lindahl et al. (1970) Science 170:447. The radioactivity bound to the disc is measured in scintillation fluid in a scintillation counter. The IC50 is determined for each putative inhibitor, for both DNA polymerases. The ratio of these IC50s determines which inhibitors are considered to be specific for DNA polymerase alpha.

SEQ ID NOs referenced herein are listed at Table 1.

TABLE 1
Sequence Identifiers

| SEQ | | |
|---|---|---|
| ID | | |
| NO | Description | Sequence or Description |
| 1 | Luciferase siRNA | CAUUCUAUCCUCUAGAGGAUGdTdT |
| 2 | Chk1 siRNA | GAAGCAGUCGCAGUGAAGAdTdT |
| 3 | Polα siRNA | GCAGUAACAUCGAUUGUAAUU |
| 4 | Polε siRNA | AGAGAAGGCUGGCGGAUUAUU |
| 5 | Polδ siRNA | CCGACGUGAUCACCGGUUAUU |
| 6 | ATR siRNA | GGUCAGCUGUCUACUGUUAUU |
| 7 | ATM siRNA | AGGAGGAGCUUGGGCCUUUUU |
| 8 | Chk1 immunogen | CNRERLLNKMCGTLPYVAPELLKRREF |
| 9 | biotinylated human | RSGLYRSPSMPENLNRPR-biotin |
| | CDC25C fragment | |
| 10 | hemagglutinin | YDVPDYAS |
| | epitope tag | |
| 11 | human DNA | DNA (NM_016937) |
| | polymerase alpha | |
| 12 | human DNA | Protein of SEQ ID NO: 11 |
| | polymerase alpha | |
| 13 | human Chk1 | DNA (NM_001274) |
| 14 | human Chk1 | Protein of SEQ ID NO: 13 |

1. A method of treating a proliferative disorder comprising:
inhibiting the activity of DNA polymerase alpha; and
inhibiting the activity of at least one checkpoint kinase.

2. The method of claim 1 wherein the checkpoint kinase is Chk1.

3. A method of treating a proliferative disorder in a subject comprising:
administering to the subject an inhibitor of DNA polymerase alpha; and
administering to the subject an inhibitor of at least one checkpoint kinase.

4. The method of claim 3 wherein the checkpoint kinase is Chk1.

5. The method of claim 2 wherein the inhibition of DNA polymerase alpha is at least 10-fold greater than the inhibition of DNA polymerase epsilon.

6. The method of claim 4 wherein the DNA polymerase alpha inhibitor is selected from the group consisting of 4-hydroxy-17-methylincisterol, a galactosyldiacylglycerol, cephalomannine, dehydroaltenusin, 6-(p-n-butylanilino)uracil and N2-(p-butylphenyl)guanine.

7. The method of claim 6, wherein the DNA polymerase alpha inhibitor is selected from the group consisting of cephalomannine and dehydroaltenusin.

8. The method of claim 4, wherein the Chk1 inhibitor is selected from the group consisting of pyrazofopyrimidines, imidazopyrazines, UCN-01, indolcarbazole compounds, Go6976. SB-218078, staurosporine, ICP-1, CEP-3891, isogranulatimide, debromohymenialdisine (DBH), pyridopyrimidine derivatives, PD0166285, scytonemin, diaryl ureas, benzimidazole quinolones, CHR 124, CHR 600, tricyclic diazopinoindolones, PF-00394691, furanopyrimidines, pyrrolopyrimidines, indolinones, substituted pyrazines, compound XL844, pyrimidinylindazolyamines, aminopyrazoles, 2-ureidothiophenes, pyrimidines, pyrrolopyrimidines, 3-ureidothiophenes, indenopyazoles, triazlones, dibenzodiazepinones, macrocyclic ureas, pyrazoloquinoloines, and the peptidomimetic CBP501.

9. The method of claim 8, wherein the Chk1 inhibitor is selected from the group consisting of a pyrazolopyrimidine or an imidazopyrazine.

10. The method according to claim 4 further comprising administering to the subject an anti-cancer agent selected from the group consisting of a uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, leucovirin, oxaliplatin (Eloxatin® from Sanofi-Synthelabo Pharmaceuticals, France), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17a-ethinylestradiol, diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, Avastin® (trastuzumab), Herceptin®, Bexxar®, Velcade®, Zevalin®, Trisenox®, Xeloda®, vinorelbine, profimer, Erbitux®, liposomal, thiotepa, altretamine, melphalan, lerozole, fulvestrant, exemestane, fulvestrant, ifosfomide, Rituxan® (rituximab), C225 and Campath®.

11. The method of claim 1 wherein the proliferative disorder is cancer, autoimmune disease, viral disease, fungal disease, neurological/neurodegenerative disorder, arthritis, inflammation, anti-proliferative disease, neuronal disease, alopecia, cardiovascular disease or sepsis.

12. The method of claim 11, wherein the disease is cancer.

13. The method of claim 12, wherein the cancer is selected from the group consisting of: cancer of the bladder, breast, colon, kidney, liver, lung, small cell lung cancer, non-small cell lung cancer, head and neck, esophagus, gall bladder, ovary, pancreas, stomach, cervix, thyroid, prostate, and skin, squamous cell carcinoma; leukemia, acute lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, Burkett's lymphoma; acute and chronic myelogenous leukemia, myelodysplastic syndrome, promyelocytic leukemia; fibrosarcoma, rhabdomyosarcoma; astrocytoma, neuroblastoma, glioma and schwannomas;
melanoma, seminoma, teratocarcinoma, osteosarcoma, xenoderoma pigmentosum, keratoctanthoma, thyroid follicular cancer and Kaposi's sarcoma.

14. The method of claim 1 further comprising radiation therapy.

15. A composition for the treatment of proliferative disorder, comprising:
a DNA polymerase alpha inhibitor; and
a Chk1 inhibitor.

16. The method of claim 1 further comprising:
determining whether the proliferative disorder in the subject involves reduction or loss of function of the p53 or Rb gene products; and
administering said inhibitors only to subjects in whom the proliferative disorder to be treated involves reduction or loss of function of at least one of the p53 or Rb gene products.

17. The method of claim 16 wherein the reduction or loss of function relates to the p53 gene product.

18. The method of claim 16 wherein the reduction or loss of function relates to the Rb gene product.

19. The method of claim 4 wherein the inhibitor of the activity of DNA polymerase alpha has an IC50 for DNA polymerase alpha that is at least 5-fold lower than its IC50 for DNA polymerase epsilon.

20. The method of claim 4 wherein the inhibitor of the activity of Chk1 has an 1050 for Chk1 that is at least 5-fold lower than its 1050 for CDK2.

21. The method of claim 4 wherein the inhibitor of the activity of DNA polymerase alpha is an siRNA.

22. The method of claim 4 wherein the inhibitor of the activity of DNA polymerase alpha is an antisense nucleic acid.

23. The method of claim 4 wherein the inhibitor of the activity of DNA polymerase alpha is an antibody or antigen binding fragment thereof.

24. The method of claim 4 wherein the inhibitor of the activity of Chk1 is an siRNA.

25. The method of claim 4 wherein the inhibitor of the activity of Chk1 is an antisense nucleic acid.

26. The method of claim 4 wherein the inhibitor of the activity of Chk1 is an antibody or antigen binding fragment thereof.

27. (canceled)
